(19) United States
(12) Patent Application Publication
(10) Pub. No.: US 2023/0384292 A1
(43) Pub. Date: Nov. 30, 2023

(54) PERISTALTIC PROPULSION DEVICE AND SYSTEM AND METHODS OF USE THEREOF

(71) Applicants: UNIVERSITY OF FLORDIA RESEARCH FOUNDATION, INCORPORATED, Gainesville, FL (US); ENTRINSIC, LLC, Norwood, MA (US)

(72) Inventors: Sadasivan VIDYASAGAR, Gainesville, FL (US); Anusree SASIDHARAN, Gainesville, FL (US); Astrid GROSCHE, Gainesville, FL (US); ASHOK V. KUMAR, Gainesville, FL (US); BREMAN ANIL PEETHAMBAR, Norwood, MA (US); Keerthi VARIER, Norwood, MA (US); Arun HIREGANGE, Norwood, MA (US)

(21) Appl. No.: 18/194,632

(22) Filed: Apr. 1, 2023

Related U.S. Application Data
(60) Provisional application No. 63/326,889, filed on Apr. 3, 2022.
(60) Provisional application No. 63/326,887, filed on Apr. 3, 2022.

(51) Int. Cl. G01N 33/50; A61B 5/11; B01L 3/00

(52) U.S. Cl. CPC G01N 33/5082; A61B 5/1108; B01L 3/502; B01L 2300/0654; B01L 2400/0481

(57) ABSTRACT

A system including a tissue bath; a portion of intestine positioned within the tissue bath, the portion of intestine having a first end and a second end; a pump coupled to the first end of the portion of the intestine and configured to pump a fluid through the portion of intestine; a light source positioned to illuminate the portion of intestine, a camera positioned to capture a stream of images showing movement of the portion of intestine; and a computing system coupled to the camera, the computing system including: a processor, and a memory storing instructions causing the processor to: receive the stream of images, identify a location along the portion of intestine in each image of the stream of images in real time, and measure an edge width at the location along the portion of intestine in each image of the stream of images in real time.

### CROSS-REFERENCE TO RELATED APPLICATION

This application is a Section 111(a) application relating to and claiming the benefit of commonly-owned, co-pending U.S. Provisional Patent Application No. 63/326,889, filed on Apr. 3, 2022 and entitled “PERISTALTIC PROPULSION DEVICE AND SYSTEM AND METHODS OF USE THEREOF,” the contents of which are incorporated herein by reference in their entirety.

### FIELD OF THE INVENTION

The exemplary embodiments relate to devices, systems, and methods for evaluating peristaltic movement in the intestine.

### BACKGROUND OF THE INVENTION

Peristalsis is a collective process of intestinal muscle contractions for the passage of food through the gastrointestinal (GI) tract. Although intestinal peristalsis has been studied using spatiotemporal mapping and intraluminal pressure measurements both in vivo and in vitro, the contribution of intraluminal pressure changes, longitudinal and circular muscle contraction and changes in fluid volume for fluid output is not fully understood.

### SUMMARY OF THE DISCLOSURE

In some embodiments, a system includes at least one amplifier module, a vision system including a multi-spectrum light, at least one infrared-LED sensor drop counter, and at least one segment of an intestine, wherein the system is configured to measure at least one of intraluminal pressure, diameter changes, longitudinal movements, fluid output, or any combination thereof in the at least one segment of the intestine.

In some embodiments, a system is configured to dynamically measure at least one of net anterograde/retrograde movement in an intestine, velocity of contractions in the intestine, fluid output at an arboral end of the intestine, or any combination thereof.

In some embodiments, a system includes a tissue bath; a portion of an intestine positioned within the tissue bath, wherein the portion of the intestine has a first end and a second end, and wherein the portion of the intestine extends in a longitudinal direction from the first end to the second end; a pump coupled to the first end of the portion of the intestine and configured to pump a fluid through the portion of intestine; a light source positioned to illuminate the portion of the intestine, a camera positioned to capture a stream of images showing movement of the portion of the intestine when illuminated by the light source; and a computing system coupled to the camera, wherein the computing system includes: a processor, and a non-transitory memory storing instructions which, when executed by the processor, cause the processor to: receive the stream of images, identify a location along the portion of intestine in each image of the stream of images in real time, and measure an edge width at the location along the portion of intestine in each image of the stream of images in real time.

In some embodiments, the tissue bath contains an isotonic solution.

In some embodiments, the light source is configured to emit a green light.

In some embodiments, the camera has a resolution of at least 10 megapixels.

In some embodiments, the pump is a peristaltic pump. In some embodiments, the peristaltic pump is configured to provide a flow rate that is in a range of from 0.001 milliliters per minute to 0.2 milliliters per minute.

In some embodiments, the system also includes a pressure transducer that is coupled to the portion of the intestine so as to measure an intraluminal pressure within the portion of the intestine.

In some embodiments, the system also includes a flow rate sensor that is coupled to the second end of the portion of the intestine so as to measure an outflow rate.

In some embodiments, the portion of the intestine is a portion of a mammal intestine.

In some embodiments, the instructions, when executed by the processor, cause the processor to execute pattern tracking software to measure the edge width.

In some embodiments, the instructions, when executed by the processor, further cause the processor to identify longitudinal contractions within the portion of the intestine.

In some embodiments, a system includes a tissue bath; a portion of an intestine positioned within the tissue bath, wherein the portion of the intestine has a first end and a second end, and wherein the portion of the intestine extends in a longitudinal direction from the first end to the second end; a pump coupled to the first end of the portion of intestine and configured to pump a fluid through the portion of the intestine; a light source positioned to illuminate the portion of intestine; at least one camera positioned to capture a corresponding at least one stream of images showing movement of the portion of the intestine when illuminated by the light source; a pressure transducer coupled to the portion of the intestine so as to detect an intraluminal pressure within the portion of the intestine; and a computing system coupled to the at least one camera and to the pressure transducer, wherein the computing system includes: a processor, and a non-transitory memory storing instructions which, when executed by the processor, cause the processor to: receive the at least one stream of images, identify a plurality of locations along the portion of the intestine in each image of the at least one stream of images in real time, measure an edge width at each of the plurality of locations along the portion of the intestine in each image of the stream of images in real time; and identify contractions in the portion of the intestine based on the measured edge widths at each of the plurality of locations.

In some embodiments, the light source is configured to emit a green light.

In some embodiments, the camera has a resolution of at least 10 megapixels.

In some embodiments, the system also includes a pressure transducer that is coupled to the portion of the intestine so as to measure an intraluminal pressure within the portion of the intestine.

In some embodiments, the system also includes a flow rate sensor that is coupled to the second end of the portion of the intestine so as to measure an outflow rate.

In some embodiments, the portion of the intestine is a portion of a mammal intestine.

In some embodiments, the contractions include at least one of longitudinal contractions or circular contractions.

In some embodiments, a method includes operating a system including: a tissue bath; a portion of an intestine positioned within the tissue bath, wherein the portion of the intestine has a first end and a second end, and wherein the portion of the intestine extends in a longitudinal direction from the first end to the second end; a pump coupled to the first end of the portion of intestine and configured to pump a fluid through the portion of the intestine; a light source positioned to illuminate the portion of the intestine; at least one camera positioned to capture a corresponding at least one stream of images showing movement of the portion of the intestine; a pressure transducer coupled to the portion of intestine so as to measure an intraluminal pressure within the portion of the intestine; and a computing system coupled to the at least one camera and to the pressure transducer, wherein the computing system includes: a processor, and a non-transitory memory storing instructions which, when executed by the processor, cause the processor to: receive the at least one stream of images, identify a plurality of locations along the portion of the intestine in each image of the at least one stream of images in real time, measure an edge width at each of the plurality of locations along the portion of the intestine in each image of the stream of images in real time; and identify contractions in the portion of the intestine based on the measured edge widths at each of the plurality of locations; providing a first formulation, wherein the first formulation includes an active ingredient in a solution; utilizing the pump to pump the first formulation through the portion of the intestine; receiving, by the computing system, the at least one stream of images showing movement of the portion of the intestine while the first formulation is pumped through the portion of the intestine; identifying, by the computing system, the plurality of locations along the portion of the intestine in each image of the at least one stream of images in real time; measuring, by the computing system, the edge width at each of the plurality of locations along the portion of the intestine in each image of the stream of images in real time; identifying, by the computing system, contractions in the portion of the intestine based on the measured edge widths at each of the plurality of locations; determining at least one metric associated with the contractions; obtaining at least one benchmark metric associated with a second formulation; and determining an efficacy of the first formulation based on a comparison of the at least one metric to the at least one benchmark metric.

In some embodiments, the metric is a change in net anterograde/retrograde movement.

In some embodiments, a method includes receiving, by at least one processor, a plurality of images from at least one image sensor, wherein the plurality of images depicts movement of an intestinal segment at a plurality of times; receiving, by the at least one processor, a plurality of pressure measurements from at least one pressure transducer associated with the intestinal segment, wherein the plurality of pressure measurements correspond to the plurality of times; determining, by the at least one processor, a pattern of fluid flow through the intestinal segment based at least in part on the plurality of images and the plurality pressure measurements, wherein the pattern of fluid flow comprises at least one of: at least one first metric for a direction of the fluid flow, or at least one second metric for a volume of the fluid flow; determining, by the at least one processor, at least one treatment protocol based on the pattern of fluid flow; and generating, by the at least one processor, at least one recommendation to a healthcare provider for the at least one treatment protocol.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A shows a representative segment of an intestine.

FIG. 1B shows a schematic of an exemplary system.

FIG. 2A shows a sketch approximating an image of an exemplary longitudinal tracker in an exemplary intestine segment.

FIG. 2B shows a plot of longitudinal contractions against time.

FIG. 3A shows images of exemplary intestine segments with exemplary horizontal trackers shown.

FIG. 3B shows a plot of intraluminal volume against time.

FIG. 4A shows a front view photograph of an experimental setup.

FIG. 4B shows a side view photograph of an experimental setup.

FIG. 4C shows a photograph of a tissue bath of an experimental setup.

FIG. 4D shows a photograph of a data readout of an experimental setup.

FIG. 4E shows a drop counter of an experimental setup.

FIG. 4F shows a plot of flow rate against time as measured by an experimental setup.

FIG. 4G shows a vision controller of an experimental setup.

FIG. 4H shows horizontal trackers of an experimental setup.

FIG. 4I shows a longitudinal tracker of an experimental setup.

FIG. 5A shows a trend view of intraluminal pressure recorded using an experimental setup.

FIG. 5B shows a detailed view of intraluminal pressure recorded using an experimental setup.

FIG. 6A shows diameter changes measured by horizontal trackers of an experimental setup.

FIG. 6B shows anterograde-retrograde movement as calculated using an experimental setup.

FIG. 7A shows a detailed view of longitudinal contractions measured by an experimental setup.

FIG. 7B shows mean changes in longitudinal contractions measured using an experimental setup.

FIG. 7C shows changes in longitudinal contractions over time measured using an experimental setup.

FIG. 8A shows correlation between fluid output, intraluminal volume, intraluminal pressure, and longitudinal contractions measured using an experimental setup.

FIG. 8B shows changes in pressure, volume, and longitudinal contractions measured using an experimental setup.

FIG. 9 shows a table of various data recorded using an experimental setup.

FIG. 10 shows classification of fluid output based on data recorded using an experimental setup.

FIG. 11 shows a graph of data recorded using experimental setup, showing the correlation between longitudinal muscle movement, intraluminal pressure, and droplet detection over a given time period.

FIG. 12 shows a graph of data recorded using experimental setup, showing the correlation between intraluminal volume, intraluminal pressure, and droplet detection over a given time period.

FIG. 13A shows a drawing of a representative segment of intestine at a first stage of peristalsis.

FIG. 13B shows a drawing of the representative segment of intestine shown in FIG. 13A, the segment of intestine being shown at a second stage of peristalsis.

### DETAILED DESCRIPTION OF THE DRAWINGS

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

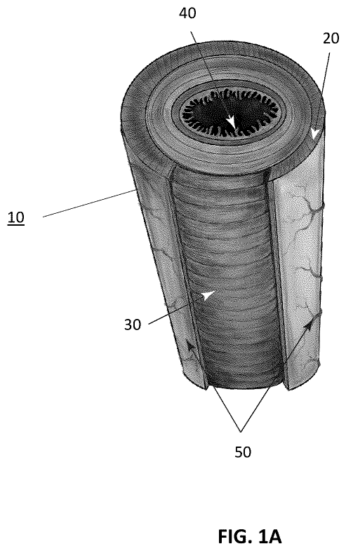

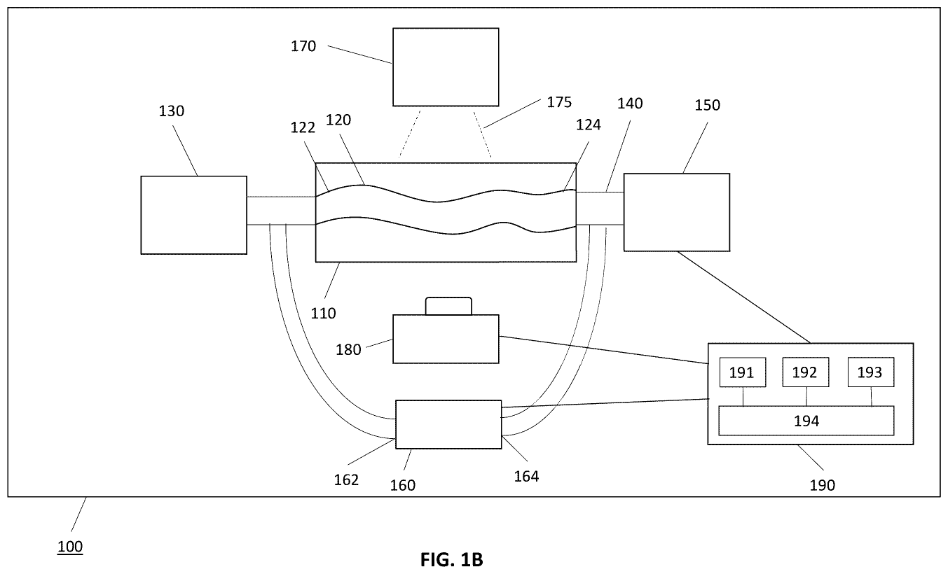

Although peristalsis has been extensively studied using spatiotemporal mapping and pressure transducers both in vivo and in vitro, the quantitative analysis of peristaltic propulsion is not fully understood. Lack of a definitive tool to study peristaltic propulsion has limited drug discovery in the field of constipation, including that associated with irritable bowel syndrome with constipation (IBS-C). The exemplary embodiments provide a device and system that utilize advanced imaging techniques and computers capable of high-speed processing in combination with multiple high-resolution, high-speed precision cameras and high-speed control of directional lighting on single controllers to achieve a fast image reproduction rate sufficient to investigate peristaltic propulsion in real time. As described herein, the exemplary embodiments utilize directional lighting and advanced field-imaging techniques together with intraluminal pressure recordings to determine intestinal diameter, frequency of longitudinal and circular muscle activity, and pressure changes, as well as velocity of anterograde and retrograde contractions and volume output measurements, to thereby determine the net peristaltic activity. FIG. 1A shows a cutaway illustration of a representative segment of intestine 10. As shown in FIG. 1A, the intestine 10 includes longitudinal muscles 20 and circular muscles 30 (shown exposed by a cutaway portion of the longitudinal muscles 20) surrounding an internal lumen 40 formed by mucosa with villi. The exterior of the longitudinal muscles 20 is surrounded by a serosal layer 50 including blood vessels. In some embodiments, anterograde, segmental, and retrograde contractions are assessed based on intestinal diameter and progression of contraction waves over time along the intestine, and are transformed into a computer-implemented interpretable cohesive readout using a computer-implemented process. The sum of all these contractions, as determined in accordance with the exemplary embodiments described herein, can be used to help determine which agent or formulation would result in net anterograde contraction (forward propulsive movement of the contents of the lumen), that would in turn lead to an increased intestinal outflow. The exemplary devices, systems, and methods described herein also reveal relative contributions of intraluminal pressure changes, longitudinal and circular muscle contraction, and changes in fluid volume to fluid output.

The exemplary embodiments include computer-implemented processes for calculation of various aspects of intraluminal pressure, longitudinal contraction, intestinal diameter, and real time volume changes to generate a computer-implemented interpretable cohesive readout which contributes to a final determination of intestinal output. The exemplary embodiments provide first-in-class devices, systems, and methods that can accurately measure various events in the intestine and generate, via computer-implemented algorithms, interpretable cohesive readouts to determine actual outflow. The patterns calculated using the exemplary processes enable one to clearly determine the outflow from retrograde, segmental, and anterograde contractions, and to determine how longitudinal contractions influence positively or negatively intestinal outflow.

The exemplary embodiments described herein relate to a device and system configured to accurately measure peristaltic activity in the GI tract in real time and to analyze and correlate these accurate measurements of peristaltic activity to generate a computer-implemented interpretable cohesive readout/s via computer-implemented algorithms, with which one can determine actual GI outflow. Prior to the discovery of the present inventors, actual GI outflow could not be determined accurately. The exemplary devices and systems and methods using same make possible accurate measurements of real time oscillations of the various muscle layers of the GI tract and predict their contribution to outflow, an objective that has not been achieved in the past. Accordingly, exemplary embodiments described herein provide for the first time a device and system that can be used to screen candidate agents to determine their ability to modulate various muscle layers specifically, and thereby to regulate peristaltic activity.

Results presented herein reveal that longitudinal muscle activity, circular muscle activity, and intraluminal pressure (ILP) oscillations have a similar frequency, suggesting a common pacemaker activity in the intestine. The discovery of a common pacemaker provides a therapeutic target against which candidate agents can be specifically screened. Results presented herein also show that an increase in intraluminal pressure (P1) is required together with high or low amplitude longitudinal and circular muscle activity for net intestinal outflow. The results also reveal that net luminal flow can be passive or active based on the contribution of ILP and longitudinal and circular muscle activity and that only active contractions result in intestinal outflow. In combination, results presented herein show that net anterograde/retrograde movement, the velocity of contractions, and the fluid output of the GI can be measured with accuracy in real-time and correlated and transformed into a computer-implemented interpretable cohesive readout that can be used to understand mechanistic contributors to peristalsis, which could potentially lead to the development of therapeutic agents for conditions that manifest with GI dysfunction. Such conditions include, for example, constipation associated with irritable bowel syndrome with constipation (IBS-C) and intestinal discomfort arising segmental contractions (which mix, separate, and churn the intestinal contents in a localized area) absent changes in longitudinal and circular muscle activity and ILP that propel the intestinal contents forward.

In some embodiments, a target for therapeutic interventions such as those described above is to screen for and identify a therapeutic agent or combination of therapeutic agents, administration of which would lead to more coordinated muscle contractions and activity ILP oscillations, thereby promoting and/or restoring coordinated GI function to achieve smooth controlled bowel movements. The exemplary embodiments described herein can be used to identify such therapeutic agents and/or combinations of therapeutic agents that can be administered to subjects afflicted by conditions that manifest with GI dysfunction, thereby treating the subjects. Accordingly, therapeutic agents and/or combinations of therapeutic agents identified using the exemplary devices, systems, and/or methods described herein can be used in methods to treat subjects afflicted by conditions that manifest with GI dysfunction. Use of therapeutic agents and/or combinations of therapeutic agents identified using the exemplary devices, systems, and/or methods in the treatment of conditions that manifest with GI dysfunction and in the preparation of medicaments for use in the treatment of conditions that manifest with GI dysfunction are also envisioned.

Other exemplary embodiments are directed to such therapeutic agents or therapeutic formulations screened or identified by the devices, systems, and/or methods described herein, where such agents or formulations administered to a subject are capable of modulating various intestinal muscle layers, regulating peristaltic activity, treating a subject afflicted by conditions that manifest with a GI dysfunction (e.g., dysfunction in peristalsis), or the like, or any combinations thereof. In some aspects, such agents or formulations identified by the devices, systems, and/or methods described herein are used to treat a subject suffering from a symptom of constipation, including but not limited to diseases or conditions such as irritable bowel syndrome with constipation (IBS-C), where a therapeutically effective amount of the agent or the formulation is administered to the subject. The therapeutically effective amount of the agent or the formulation is an amount sufficient to reverse, essentially reverse, or improve the GI dysfunction or the dysfunction in peristalsis in the subject, thereby treating the subject suffering such an affliction, such as constipation or IBS-C. In other aspects, also provided are uses of such therapeutic agents or therapeutic formulations in the manufacture of a medicament for the treatment of constipation, including IBS-C. Further aspects of the disclosure provide for uses of such therapeutic agents or therapeutic formulations described herein as a regulator of GI dysfunction, including a regulator of peristaltic dysfunction, thereby treating a subject suffering from GI dysfunction or peristaltic dysfunction. Additional embodiments provide for methods of treating a subject suffering from GI dysfunction (e.g., dysfunction in peristalsis) by administering, to the subject, a therapeutically effective amount of the agent or the formulation screened or identified by the devices, systems, and/or methods described herein, where such agents or formulations are capable of modulating various intestinal muscle layers; regulating peristaltic activity; reversing, essentially reversing, or improving the GI dysfunction (or dysfunction in peristalsis); or any combination thereof, thereby treating the subject.

FIG. 1B schematically illustrates elements of an exemplary system 100 configured for observing peristaltic action of the GI tract. In some embodiments, the system 100 includes a tissue bath 110. In some embodiments, the tissue bath 110 is a horizontal tissue bath. In some embodiments, the tissue bath 110 is a water-jacketed plexiglass tissue bath. In some embodiments, the tissue bath 110 is of the type commercialized by Harvard Apparatus of Holliston, Massachusetts under the trade name MAYFLOWER HORIZONTAL TISSUE BATH. In some embodiments, the tissue bath 110 contains an isotonic solution. In some embodiments, the tissue bath 110 contains Ringer's solution.

Continuing to refer to FIG. 1B, in some embodiments, the system 100 includes an intestine segment 120 (e.g., a portion of an intestine) within the tissue bath 110. In some embodiments, the intestine segment 120 has a proximal end 122 (e.g., the end of the intestine segment 120 that is closer to the mouth when the intestine segment 120 is in the body) and a distal end 124 (e.g., the end of the intestine segment 120 that is closer to the anus when the intestine segment 120 is in the body) opposite the proximal end 122. In some embodiments, the intestine segment 120 is a portion of a mammal intestine. In some embodiments, the intestine segment 120 is a portion of a rodent intestine. In some embodiments, the intestine segment 120 is a portion of a mouse intestine. In some embodiments, the intestine segment 120 is a portion of a primate intestine. In some embodiments, the intestine segment 120 is a portion of a human intestine. In some embodiments, the intestine segment 120 is between 1 centimeter and 10 centimeters in length. In some embodiments, the intestine segment 120 is between 2.5 centimeters and 7.5 centimeters in length. In some embodiments, the intestine segment 120 is about 5 centimeters in length. In some embodiments, the intestine segment 120 is 5 centimeters in length. In some embodiments, the intestine segment 120 is a portion of a small intestine. In some embodiments, the intestine segment 120 is a jejunum segment, a mid-jejunum segment, an ileum segment, or a colon segment.

In some embodiments, the system 100 includes a pump 130 coupled to the proximal end 122 of the intestine segment 120. In some embodiments, the pump 130 is a peristaltic pump. In some embodiments, the pump 130 is a peristaltic roller pump. In some embodiments, the pump 130 is configured to pump a fluid into the proximal end 122 of the intestine segment 120. In some embodiments, the fluid includes a test sample. In some embodiments, the test sample includes a substance that is being tested to evaluate its effect on the intestine as described above. In some embodiments, the pump 130 is configured to pump the fluid at a constant flow rate. In some embodiments, the flow rate is in a range of between 0.001 mL/minute and 0.2 mL/minute. In some embodiments, the flow rate is between 0.001 mL/minute and 0.15 mL/minute. In some embodiments, the flow rate is between 0.001 mL/minute and 0.1 mL/minute. In some embodiments, the flow rate is between 0.03 mL/minute and 0.2 mL/minute. In some embodiments, the flow rate is between 0.03 mL/minute and 0.15 mL/minute. In some embodiments, the flow rate is between 0.03 mL/minute and 0.1 mL/minute. In some embodiments, the flow rate is between 0.05 mL/minute and 0.1 mL/minute. In some embodiments, the flow rate is between 0.05 mL/minute and 0.08 mL/minute. In some embodiments, the flow rate is between 0.06 mL/minute and 0.07 mL/minute. In some embodiments, the flow rate is about 0.065 mL/minute and 0.2 mL/minute. In some embodiments, the flow rate is 0.065 mL/minute. In some embodiments, the system 100 includes a further pump that is coupled to the tissue bath 110 extraluminally (e.g., outside the lumen defined by the intestine segment 120) and is configured to pump a fluid outside the intestine segment 120.

In some embodiments, the system 100 includes an outlet 140 coupled to the distal end 124 of the intestine segment 120. In some embodiments, the outlet 140 is maintained at a constant pressure in order to provide consistent flow conditions for the intestine segment 120. In some embodiments, the constant pressure is between 0.1 cm of H 2 O and 10 cm of H₂O. In some embodiments, the constant pressure is between 0.1 cm of H 2 O and 3 cm of H₂O. In some embodiments, the constant pressure about 1.5 cm of H₂O. In some embodiments, the constant pressure is 1.5 cm of H₂O. In some embodiments, the outlet 140 is maintained at a constant pressure by positioning the outlet 140 at a suitable distance above the intestine segment 120. For example, in some embodiments in which the constant pressure is 1.5 cm of H₂O, the outlet 140 is positioned 1.5 cm above the intestine segment 120.

In some embodiments, the system 100 includes a flow sensor 150 that is fluidically coupled to the outlet 140. In some embodiments, the flow sensor 150 is a drop counter. In some embodiments, the flow sensor 150 is an infrared LED drop counter. In some embodiments, the flow sensor 150 is an infrared LED drop counter of the type commercialized by Vernier Science Education of Beaverton, Oregon under the trade name GO DIRECT. In some embodiments, the flow sensor 150 is an infrared LED drop counter that is configured such that fluid passing through the outlet blocks LED light falling on a detector of the LED drop counter, thereby enabling the volume of such fluid to be measured.

In some embodiments, the system 100 includes a pressure sensor 160 that is coupled to the intestine segment 120 so as to measure intraluminal pressure within the intestine segment 120. In some embodiments, the pressure sensor 160 is a pressure transducer. In some embodiments, the pressure sensor 160 includes a piezoresistor. In some embodiments, the piezoresistor is a monolithic piezoresistor. In some embodiments, the pressure sensor includes a monolithic piezoresistor that is ion implanted on a silicon diaphragm. In some embodiments, the pressure sensor 160 provides high sensitivity (e.g., sensitivity of 0.1 mV/mbar). In some embodiments, the pressure sensor 160 provides low temperature drift (e.g., within ±0.03 mbar/° C.). In some embodiments, the pressure sensor 160 includes two pressure ports 162, 164. In some embodiments, the pressure sensor 160 includes a “+” pressure port for an after load/aboral end, and a “−” pressure port for a negative pressure/anal oral end.

In some embodiments, the system 100 includes a light source 170 producing light 175 to illuminate the intestine segment 120. In some embodiments, the light source 170 is a multi-spectrum light source. In some embodiments, the light source 170 is a multi-spectrum light source of the type commercialized by Keyence Corporation of America of Itasca, Illinois under the trade name LUMITRAX. In some embodiments, the light source 170 is configured to produce light of a predetermined wavelength and intensity. In some embodiments, the light is red light. In some embodiments, the light is blue light or violet light. In some embodiments, use of violet light has a stimulating effect on movement of the intestine segment 120. In some embodiments, the predetermined wavelength and intensity are selected so as to provide sufficient contrast in the observed appearance of arteries within the intestine segment 120 to thereby perform the image-based analysis that will be described hereinafter. In some embodiments, the predetermined wavelength and intensity are selected so as not to stimulate muscles of the intestine segment 120, which would influence the analysis that will be described hereinafter. In some embodiments, the light is green light. In some embodiments, the light has a wavelength of between 495 nanometers and 570 nanometers. In some embodiments, the green light (e.g., having a wavelength of between 495 nanometers and 570 nanometers) provides sufficient contrast in the observed appearance of arteries within the intestine segment 120, while not stimulating muscles of the intestine segment 120.

In some embodiments, the system 100 includes a camera 180 imaging the intestine segment 120. In some embodiments, the camera 180 captures a stream of images (e.g., a sequence of images) that depicts movement of the intestine segment 120 while the pump 130 is in operation to pump the fluid through the intestine segment 120. In some embodiments, the sequence of images is captured at a frequency of between one image per 25 milliseconds and one image per 300 milliseconds. In some embodiments, the sequence of images is captured at a frequency of between one image per 25 milliseconds and one image per 100 milliseconds. In some embodiments, the sequence of images is captured at a frequency of between one image per 25 milliseconds and one image per 75 milliseconds. In some embodiments, the sequence of images is captured at a frequency of about one image per 50 milliseconds (e.g., the camera is 180 is set up to capture images at a frequency of about one image per 50 milliseconds, but the actual rate of image capture is less frequent, such as one image per 60 milliseconds, due to processing lag or other hardware or software issues). In some embodiments, the sequence of images is captured at a frequency of one image per 50 milliseconds. In some embodiments, the camera 180 is a high-resolution camera. In some embodiments, the camera 180 has a resolution of at least 10 megapixels, e.g., between 10 megapixels and 300 megapixels. In some embodiments, the camera 180 has a resolution of at least 20 megapixels, e.g., between 20 megapixels and 300 megapixels. In some embodiments, the camera 180 has a resolution of 21 megapixels. In some embodiments, the camera 180 has a resolution of 64 megapixels. In some embodiments, the camera 180 is a high-speed camera. In some embodiments, the camera 180 has an image transfer time of less than 30 milliseconds, e.g., an image transfer time of about 20 milliseconds, or an image transfer time of 20.2 milliseconds. In some embodiments, the camera 180 is a camera of the type commercialized by Keyence Corporation of America of Itasca, Illinois under the trade name CV-X, such as the camera of the model number CV-X400.

In some embodiments, the system 100 includes a computing device 190 for evaluating peristaltic performance in accordance with one or more embodiments of the present disclosure. In some embodiments, the computing device 190 may include hardware components such as a processor 191, which may include local or remote processing components. In some embodiments, the processor 191 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor 191 may include data-processing capacity provided by the microprocessor. In some embodiments, the microprocessor may include memory, processing, interface resources, controllers, and counters. In some embodiments, the microprocessor may also include one or more programs stored in memory.

Similarly, the computing device 190 may include storage 192, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage 192 may include, e.g., a suitable non-transient computer readable medium such as, e.g., random access memory (RAM), read only memory (ROM), one or more buffers and/or caches, among other memory devices or any combination thereof.

In some embodiments, computing device 190 may implement computer engines for evaluating peristaltic performance in accordance with the exemplary embodiments described herein. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; ×86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, to evaluate peristaltic performance, the computing device may include computer engines including, e.g., a peristalsis analysis service 193. In some embodiments, the peristalsis analysis service 193 may include dedicated and/or shared software components, hardware components, or a combination thereof. For example, the peristalsis analysis service 193 may include a dedicated processor and storage. However, in some embodiments, the peristalsis analysis service 193 may share hardware resources, including the processor 191 and storage 192 of the computing device 190 via, e.g., a bus 194. Thus, the peristalsis analysis service 193 may include a memory (e.g., a non-transitory memory) including software and software instructions executable by a processor and which, when executed by the processor, cause the processor to perform processes such as, e.g., machine learning models and/or logic for implementing analysis of peristaltic performance.

In some embodiments, the computing device 190 is communicatively coupled (e.g., via a wired connection or a wireless connection) to the flow sensor 150, to the pressure sensor 160, and to the camera 180, to receive data recorded thereby (e.g., to receive output flow rate data captured by the flow sensor 150, intraluminal pressure data captured by the pressure sensor 160, and images of the intestine segment 120 captured by the camera 180).

The exemplary system 100 described above includes one of each of the various elements described therein (e.g., one of the tissue bath 110, one of the intestine segment 120, one of the pump 130, etc.). In other embodiments, an exemplary system 100 includes more than one of some or all of the elements described above. For example, in some embodiments, an exemplary system 100 includes four separate ones of the intestine segment 120, e.g., one each of a jejunum segment, a mid-jejunum segment, an ileum segment, and a colon segment, and includes four separate ones of the tissue bath 110, each of which holds one of the intestine segments 120. In some such embodiments, an exemplary system 100 also includes four separate ones of the pump 130, the outlet 140, the flow sensor 150, the pressure sensor 160, the light source 170, and/or the camera 180, one for each one of the intestine segment 120.

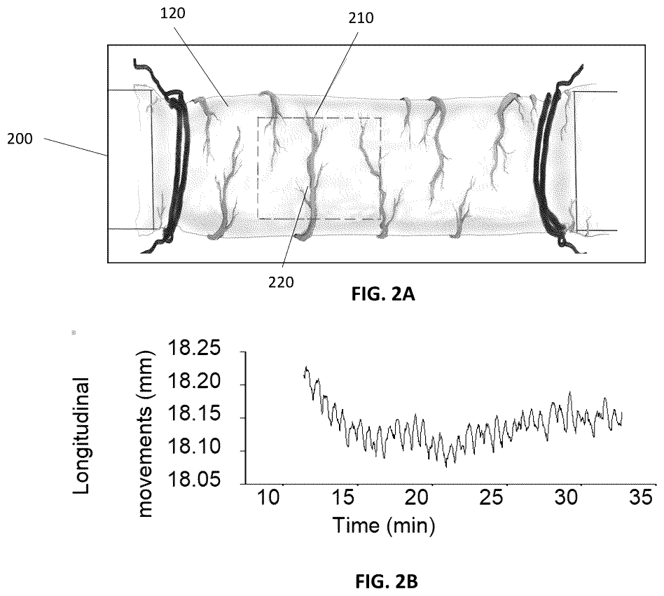

In some embodiments, the computing device 190 is configured to identify and monitor trackers in images obtained from the camera 180 to thereby identify longitudinal contractions within the intestine segment 120. In some embodiments, an artery having a specific pattern is marked within an image captured by the camera 180 and longitudinal movement of the pattern (e.g., movement of the pattern in a direction along the length of the intestine segment 120) is tracked to thereby identify longitudinal contractions within the intestine segment 120. In some embodiments, the artery is marked manually (e.g., by a user). In some embodiments, the computing device implements software to identify a suitable artery. In some embodiments, the computing device 190 implements pattern tracking software to measure longitudinal contractions within the intestine segment 120. In some embodiments, the pattern tracking software is the outline pattern tool commercialized by Keyence Corporation of America of Itasca, Illinois under the trade name PATTERN TRAX. FIG. 2A shows an illustration 200 approximating an image of the intestine segment 120 captured by the camera 180. In the illustration 200, a region 210 containing an appropriate artery 220 is identified. FIG. 2B shows a chart 230 of longitudinal movements with respect to time that is obtained using the method described above. In the chart 230, an upward shift indicates contraction of the intestine segment 120 and a downward shift indicates relaxation of the intestine segment 120. In some embodiments, contraction correlates with movement of a captured artery toward the proximal end 122 of the intestine segment 120 and relaxation correlates with movement of the captured artery toward a distal end 124 of the intestine segment 120.

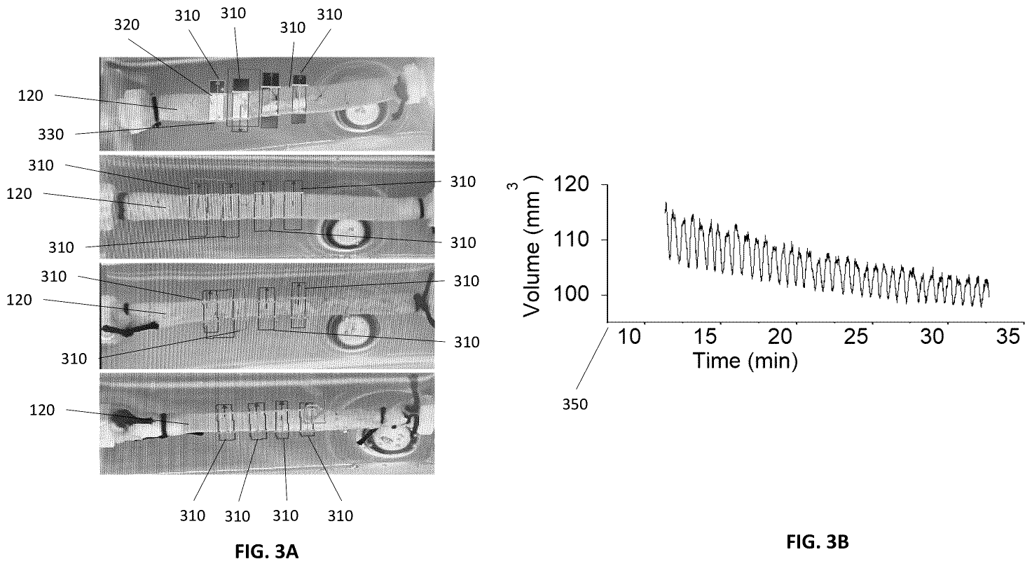

In some embodiments, the computing device 190 is configured to identify and monitor trackers in images obtained from the camera 180 to thereby computationally measure edge width and evaluate horizontal (e.g., diametric) contractions of the intestine segment 120. In some embodiments, the computing device 190 implements pattern tracking software to measure horizontal contractions within the intestine segment 120. In some embodiments, the pattern tracking software is the outline pattern tool commercialized by Keyence Corporation of America of Itasca, Illinois under the trade name PATTERN TRAX. In some embodiments, the computing device 190 measures edge width at a plurality of locations within the intestine segment 120. In some embodiments, the computing device 190 measures edge width at four locations within the intestine segment 120. FIG. 3A shows images of four exemplary intestine segments 120. In each of the intestine segments 120, four outlines 310 show regions within which edge width is to be measured. In each outline 310, a first edge 320 and a second edge 330 show detected edges of the intestine segment 120, between which the width is measured to thereby evaluate horizontal contractions. (For clarity of illustration, the first edge 320 and the second edge 330 are specifically identified with reference numerals only for a first one of the intestine segments 120.) In some embodiments, the computing device 190 is configured to measure the width at each relevant location in real time and to record a time series of width measurements. In some embodiments, edge widths are measured in captured images in terms of pixels and are converted to units of length (e.g., millimeters). In some embodiments, edge widths are converted from pixels to units of length based on a scaling ratio. In some embodiments, a scaling ratio is determined by measuring a length of the intestine segment 120 in units of length (e.g., millimeters), measuring a length of the intestine segment 120 in terms of pixels in a captured image, and determining a scaling ratio based on a ratio between the measured lengths. In some embodiments, the length of the intestine segment 120 in units of length is measured using a measurement device (e.g., vernier calipers).

In some embodiments, the computing device 190 is configured to calculate a volume within each of the intestine segments 120. In some embodiments, the computing device 190 is configured to calculate the volume based on a specialized version of the general formula V=πr²h. In this formula, the radius r is equal to half of the diameter (e.g., edge width) as measured as described above. In some embodiments, the height h is equal to the length of the segment. In some embodiments, the length is a nominal length (e.g., 5 centimeters). In some embodiments, the length is determined based on longitudinal contractions within the intestine segment 120, as described above with reference to FIGS. 2A and 2B. In some embodiments, the volume V within each of the intestine segments 120 is calculated according to the formula:

V=π[h₁(d₁/2)²+g₁(d₁/2)²+h₂(d₂/2)²+g₂(d₂/2)²+h₃(d₃/2)²+g₃(d₃/2)²+h₄(d₄/2)²]

In the above formula, d₁, d₂, d₃, and d₄ are the diameters measured by the four edge width trackers (e.g., the distance between the first edge 320 and the second edge 330 in each of the regions defined by the outlines 310 as described above), h₁, h₂, h₃, and h₄ are the lengths along the intestine segment 120 measured by each such tracker, and g₁, g₂, and g₃ are the lengths along the intestine segment 120 between adjacent ones of the trackers. In some embodiments, the computing device 190 is configured to calculate the volume within each of the intestine segments 120 and to record a time series of volumes. FIG. 3B shows an exemplary graph 350 of intraluminal volume against time that may be produced in this manner.

In some cases, raw data relating to, for example, intraluminal pressure changes, diametral (e.g., circular) and longitudinal muscle contraction, and output flow rate can be impacted adversely by the presence of signal-obfuscating noise. In some cases, such signal-obfuscating noise can the raw data largely uninterpretable. To address this issue, the exemplary embodiments include an algorithm-based computer-implemented process to filter out the signal-obfuscating noise from the raw data, thereby transforming the results into an interpretable cohesive readout. In some embodiments, the denoising process is based at least on the moving average and the standard deviation of the data being denoised. In some embodiments, the denoising process includes a configurable smoothing parameter. In some embodiments, the smoothing parameter is typically minimized to avoid data loss.

In some embodiments, the peristalsis analysis service 193 implements a synchronization process to synchronize data captured from various elements of the system 100 (e.g., the flow sensor 150, the pressure sensor 160, the camera 180, collectively referred to as the “data capture elements”). In some embodiments, each of the data capture elements includes its own integrated computing device, or is coupled to its own dedicated computing device, that effects data capture from such element, and each such computing device has its own system clock. Additionally, in some embodiments, some of the data capture elements capture data at different frequencies than others. For example, in some embodiments, the pressure sensor 160 captures data at a first frequency (e.g., one sample every 20 milliseconds) and the camera 180 captures data at a different second frequency (e.g., one image every 50 milliseconds). In some embodiments, synchronization is performed by matching the system clocks for the computing devices of each of the data capture elements at a time when a first output drop is recorded by the flow sensor 150 to the count of the pressure sensor 160 and to the camera 180 (or data derived from images captured by the camera 180).

In some embodiments, to synchronize data captured from the pressure sensor 160 (e.g., which is captured at a first time frequency, such as one sample every 20 milliseconds) with data captured from the camera 180 (e.g., which is captured at a second time frequency, such as one sample every 50 milliseconds), the following procedure is followed. In some embodiments, during acquisition of data from the camera 180, which is used to track longitudinal movements of the intestine segment 120 as described above, starting and ending points of a data sample captured by the camera 180 are matched to corresponding points in the data captured by the pressure sensor 160 in software operating the pressure sensor 160. In some embodiments, data acquired using the camera 180 is captured in the form of data counts (e.g., samples are numbered sequentially). In some embodiments, a time per count is calculated based on the total number of counts and the total length of the time window of data captured using the pressure sensor 160. In some embodiments, the time for each data sample is determined by adding the time corresponding to the starting point to the count for the data sample multiplied by the time per count. In some embodiments, the time is then converted into minutes for representation.

In some embodiments, to synchronize data captured from the pressure sensor 160 with data captured from the flow sensor 150 (e.g., a drop counter), the following procedure is followed. In some embodiments, data captured from the flow sensor 150 is in the form of cumulative time and cumulative volume. In some embodiments, the volume of each drop is assumed to be a constant, and the time for each drop is then calculated by determining the number of drops by dividing the cumulative volume by the volume per drop, and then by dividing the cumulative time by the number of drops. In some embodiments, during data acquisition, the timestamp of the data captured by the pressure sensor 160 is matched to the time of the first drop recorded by the flow sensor 150. In some embodiments, each drop is then matched to the time of the data captured by the pressure sensor 160 to synchronize pressure data captured by the pressure sensor 160 with volume data captured by the flow sensor 150.

Understanding the contribution of each of the muscle layers and pressure changes in the intestinal lumen, as enabled by the exemplary embodiments, may help better understand and lead to advances in the regulation of each of these contributors independently to better control net intestinal outflow. Moreover, the ability to modulate each of the contributors to the outflow, as enabled by the exemplary embodiments, makes it possible to fine tune therapeutic intervention to meet the specific needs of a patient. The patterns calculated in accordance with the exemplary embodiments will enable one to clearly determine the outflow from retrograde, segmental, and anterograde contractions and how longitudinal contractions will finally influence positively or negatively the intestinal outflow.

In some embodiments, the system 100 is utilized to evaluate the efficacy of a formulation at influencing intestinal outflow as follows. In some embodiments, a method includes providing a first formulation (e.g., a formulation being evaluated) including an active ingredient in a solution. In some embodiments, the pump 130 is utilized to pump the solution through the intestine segment in the manner described above with reference to the pump 130. In some embodiments, the camera 180 captures at least one stream of images showing movement of the intestine segment 120 while the first formulation is pumped through the intestine segment, and the computing device 190 receives the at least one stream of images from the camera 180. In some embodiments, the computing device 190 identifies a plurality of locations (e.g., tracker locations) along the portion of the intestine in each image of the at least one stream of images in real time, such as in the manner described above with reference to the computing device 190. In some embodiments, the computing device 190 measures an edge width at each of the plurality of locations along the portion of the intestine in each image of the stream of images in real time, such as in the manner described above with reference to the computing device 190. In some embodiments, the computing device 190 identifies contractions in the portion of the intestine based at least on the measured edge widths at each of the plurality of locations. In some embodiments, the computing device 190 determines at least one metric associated with the contractions. In some embodiments, the computing device 190 obtains at least one benchmark metric associated with a second formulation (e.g., a reference formulation or a benchmark formulation). In some embodiments, the computing device 190 determines an efficacy of the first formulation based on a comparison of the at least one metric to the at least one benchmark metric.

In some embodiments, the present disclosure provides a device and system that utilizes advanced imaging techniques and computers capable of high-speed processing in combination with multiple high-resolution, high-speed precision cameras and high-speed control of directional lighting on single controllers to achieve a fast image reproduction rate sufficient to investigate peristaltic propulsion on a level hitherto never achieved. In some embodiments, the high-speed precision cameras and high-speed control of directional lighting may be used in conjunction with intraluminal pressure recordings to determine intestinal diameter, frequency of longitudinal muscle activity, and pressure changes, as well as velocity of anterograde and retrograde contractions and volume output measurements to determine the net peristaltic activity. In some embodiments, anterograde, segmental, and retrograde contractions may be assessed based on intestinal diameter and progression of contraction waves over time along the intestine and transformed into a computer-implemented interpretable cohesive readout. The sum of all these contractions can be used to help determine which agent or formulation would result in net anterograde contraction (forward propulsive movement of the contents of the lumen), that would in turn lead to an increased intestinal outflow. The device, system, and methods described herein also reveal relative contributions of intraluminal pressure changes, longitudinal muscle contraction, and changes in fluid volume to fluid output.

In some embodiments, data may be collected form one or more cameras and/or one or more pressure transducers. In some embodiments, the camera(s) may capture images through time of an intestine and/or intestinal segment, where the images are captured in synchronization with multi-spectrum lighting. Based on each image, an edge width of the intestine/intestinal segment may be measured to determine a diameter at each point in time. Additionally, based on each image, a horizontal/longitudinal length of the intestine/intestinal segment may be measured. Based on the changes to diameter and horizontal/longitudinal length through time, volume and movement of the intestine/intestinal segment may be calculated (e.g., progression of a contraction in an oral and/or aboral direction or other progression pattern).

In some embodiments, to provide a cleaner pattern for the intestinal segment, the raw data from the high-speed cameras and pressure transducers may be pre-processed and/or cleanses. For example, in some embodiments, signal-obfuscating noise may be filtered from the raw data. In another example, erroneous data points, e.g., in images from the high-speed cameras, may be identified and removed.

In some embodiments, include amplitude and frequency analysis may be performed on the data. The diametral and longitudinal contraction patterns are complex wave forms that contain many underlying frequencies. In some embodiments, the dominant frequency and amplitude of a pattern for an intestinal segment may be determined to determine the number of contractions in a certain direction or number of pressure waves. In some embodiments, the number, amplitude, duration, and frequency of high and low amplitude changes in intraluminal pressure, diametral and longitudinal muscle activity may be calculated based on the waveform resulting from the data measured for the intestinal segment.

In some embodiments, the flow through the intestine is viscous and incompressible. Both the diametral and longitudinal can contribute to increase in outflow. Thus, by analyzing the contractions within a region of the intestine, whether the contractions are anterograde, segmental, or retrograde may be determined. This can be determined using the principles of mass and volume conservation expected in incompressible viscous flow. For example, the location of the trackers placed on the intestine to measure the contractions may be used to automatically determine the volume of the intestine being studied and compute the overall volume as a function of time. Alternatively, or in addition, the volume of one or more segments of the intestinal region being studied may be computed.

In some embodiments, the volume of the intestine and/or intestinal segment(s) may be interpreted to determine if the resultant flow is forward or reverse between any two segments of that region due to a pattern of contractions. In some embodiments, an exemplary system may count an anterograde contraction where there is a volume contraction in the segments on the oral side that may coincide with expansion of segments on the aboral side. In some embodiments, an exemplary system may count a retrograde contraction where there is a volume contraction in the segments on the aboral side that may coincide with expansion of segments on the oral side. In some embodiments, the exemplary system may identify segmental behavior where there may be contraction or expansion occurring concurrently at both ends as there may be no additional flow between the two segments due to the contraction. Similarly, the overall volume contraction or expansion over an entire region of the intestine may be used to determine an overall increase or decrease in the out flow.

In some embodiments, to analyze the contribution of intraluminal pressure changes, diametral and longitudinal muscle contraction to the changes in the output flow rate, data recorded by the pressure transducers and the high-speed cameras are used to determine the number of contractions, their frequency and amplitudes for both the diametral and longitudinal contractions. In some embodiments, the exemplary system may be used to study peristalsis and intestinal flow to understand the correlation between intraluminal pressure and the various intestinal muscle contractions, which may facilitate predicting whether the contractions are causing anterograde (forward) or retrograde (reverse) flow.

In some embodiments, the data regarding net anterograde contraction and the relative contributions of intraluminal pressure changes, longitudinal muscle contraction, and changes in fluid volume to fluid output may result in patterns correlated with the outflow from retrograde, segmental, and anterograde contractions and how longitudinal contractions influence positively or negatively intestinal outflow. As a result, in some embodiments, one or more pattern recognition algorithm may be implemented to match a pattern of contractions and fluid flow through a segment to one or more signature patterns that has been predetermined to be associated with one or more particular treatment protocols. In some embodiments, the pattern of fluid low is based, at least in part, on captured images and pressure measurements as described herein. In some embodiments, an exemplary method includes generating a recommendation to a healthcare provider for at least one treatment protocol base.

### Examples

Although intestinal peristalsis has been extensively studied using spatiotemporal mapping and intraluminal pressure measurements both in vivo and in vitro, the contribution of intraluminal pressure changes, longitudinal muscle contraction and changes in fluid volume for fluid output is not fully understood. The examples described herein incorporate multiple high-resolution, high-speed precision cameras and high-speed control of directional lighting on single controllers for fast image reproduction rate. In this study, anterograde, segmental and retrograde contractions were determined from intestinal diameter and progression of the contraction waves over time along the intestine using a proprietary program. The study also helps determine the contribution of intraluminal pressure changes, longitudinal muscle contraction and changes in fluid volume towards fluid output. The examples described herein incorporate computer-implemented processes for calculation of various aspects of intraluminal pressure, longitudinal contraction, intestinal diameter, and real time volume changes to determine intestinal output. This example system is the first of its kind that is enabled to determine various events in the intestine to actual outflow.

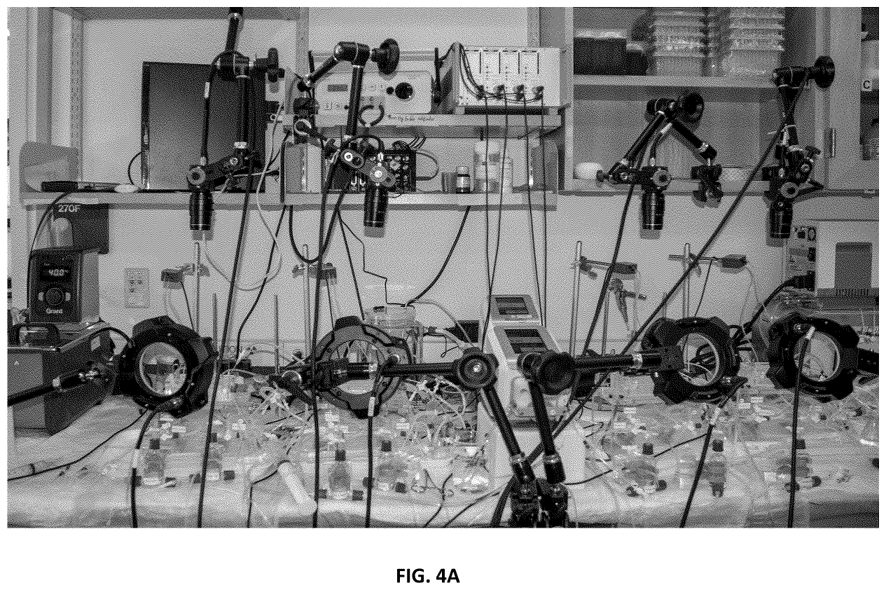

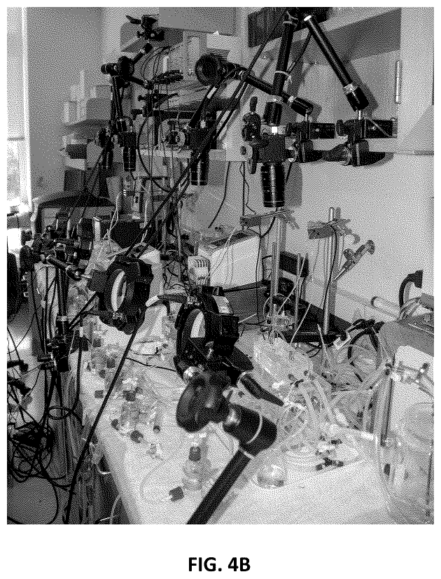

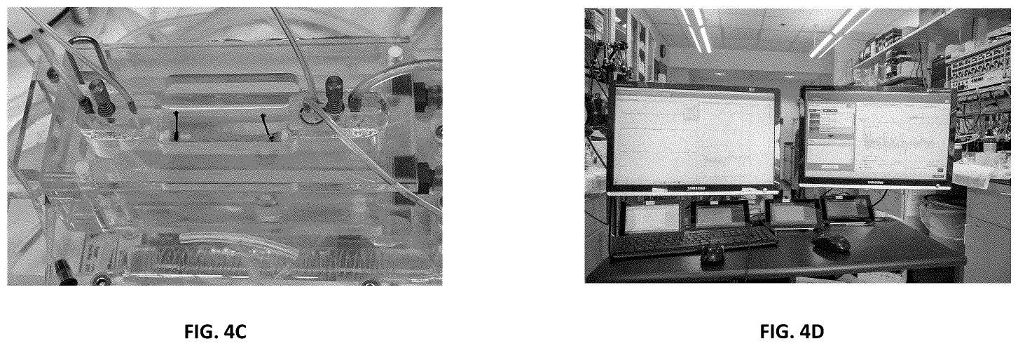

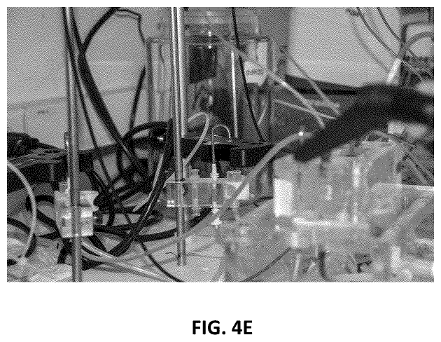

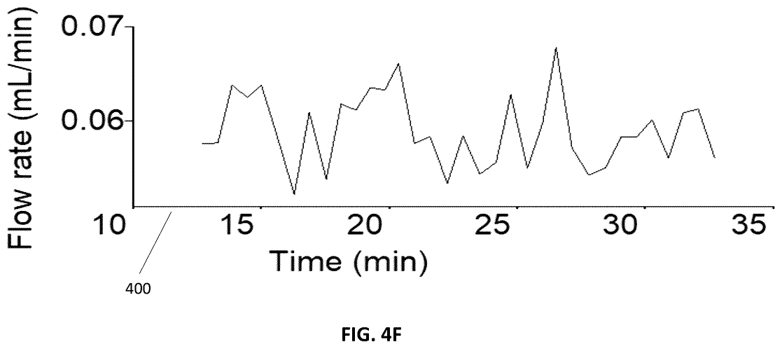

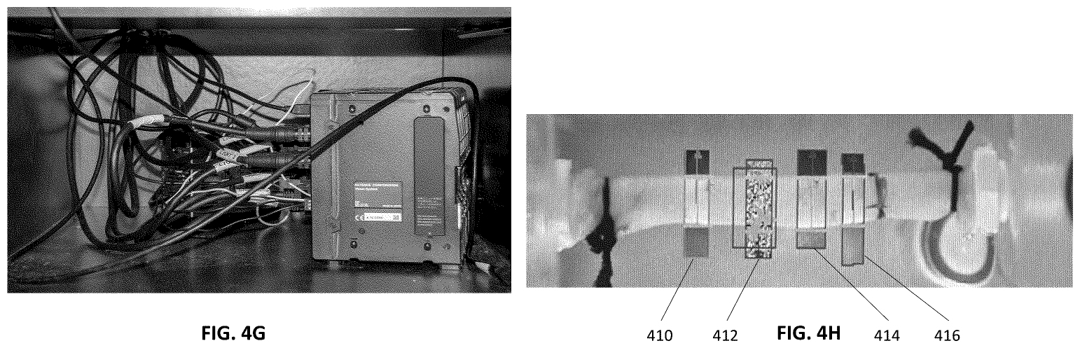

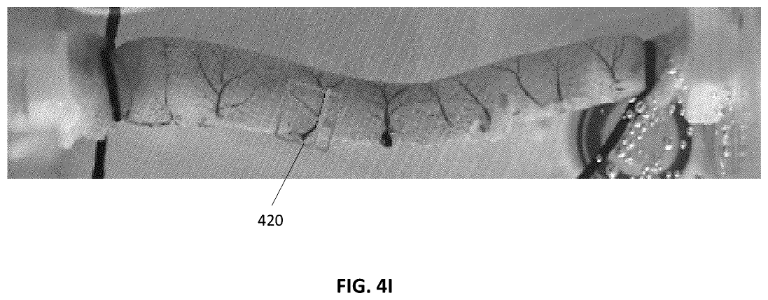

FIGS. 4A and 4B are front view and lateral view photographs, respectively, of an experimental setup for studying peristalsis. As will be described hereinafter, the experimental setup is configured to measure, for sample intestinal tissue, intraluminal pressure, intestinal diameter, longitudinal contractions, and flow rate. The experimental setup included approximately 5 cm long segments from jejunum, mid-jenumum, ileum, and colon of mouse small intestine (male, NIH Swiss). The intestine segments were mounted in a tissue bath is of the type commercialized by Harvard Apparatus of Holliston, Massachusetts under the trade name MAYFLOWER HORIZONTAL TISSUE BATH, which contained physiological Ringer's solution. The experimental setup included a peristaltic pump coupled to the proximal end of the intestine and configured to deliver a constant inflow rate of 0.065 milliliters per minute. An afterload of 1.5 cm H₂O was applied by a raised water column. The experimental setup also included pressure transducers of the type commercialized by Harvard Apparatus of Holliston, Massachusetts under the trade name DIFFERENTIAL PRESSURE TRANSDUCER MPX, which were connected to the oral and aboral end of each segment for measuring intraluminal pressure. The experimental setup also included transducer amplifier modules of the type commercialized by Harvard Apparatus of Holliston, Massachusetts under the trade name TAM-A, which were installed in a case of the type commercialized by Harvard Apparatus of Holliston, Massachusetts under the trade name PLUGSYS, and which were coupled to the pressure transducers described above. Intraluminal pressures were captured using the data acquisition software commercialized by Harvard Apparatus of Holliston, Massachusetts under the trade name BASIC DATA ACQUISITION SOFTWARE (“BDAS”). The experimental setup illuminated the intestine segments using a multi-spectrum light source of the type commercialized by Keyence Corporation of America of Itasca, Illinois under the trade name LUMITRAX. The light source was configured to emit green light having a wavelength of 520 nanometers. The experimental setup also included high-resolution and high-speed cameras of the type commercialized by Keyence Corporation of America of Itasca, Illinois under the trade name CV-X400, which were used to obtain images that were utilized to evaluate diameter changes and longitudinal movements of the experimental intestine in the manner described above. The experimental setup also included infrared-LED drop count sensors of the type commercialized by Vernier Science Education of Beaverton, Oregon under the trade name GO DIRECT, which were used in conjunction with a user interface commercialized by Vernier Science Education of Beaverton, Oregon under the trade name LABQUEST MINI to measure fluid output from the intestinal segments.

FIG. 4C is a photograph of the experimental intestinal segments perfused in a water-jacketed tissue bath as described above, and having oral and aboral ends thereof connected to pressure transducers as described above. FIG. 4D is a photograph of a display showing data visualization in a trend view and in a detailed view. FIG. 4E is a photograph of the fluid falling through a specified area of the drop-counter so that the LED light falling on the detector is blocked, resulting in the generation of a digital signal that is then captured by a data collection interface. FIG. 4F shows a graph 400 of fluid outflow (in milliliters per minute) against time, as provided by the data acquisition program as described above. Net fluid movement was determined by subtracting the input flow volume (IV) from the output flow volume (OV). OV-IV differences helped to determine secretory or the absorptive state of the intestinal segment.

FIG. 4G is a photograph of the vision controller for the high-resolution camera and multi-spectrum lightings for measuring diameter. FIG. 4H is an image of an experimental intestine sample showing tissue trackers 410, 412, 414, 416 used for measuring edge width. FIG. 4I shows tissue trackers 420 used for measuring longitudinal contractions. The present inventors determined that at least 4 trackers should be implemented in the device, system and methods described herein for accurate measurements. A horizontal tracker as described above was used to measure horizontal intestinal movements. Intraluminal volume was calculated using the formula V=πr2h.

In these experiments, the basal activity of intestinal peristalsis in terms of intraluminal pressure (ILP) changes, real-time changes in longitudinal muscle activity, and intestinal lumen diameter were measured in the manner described above using multiple tracking points in each intestinal segment. The number, strength, and amplitude of oscillations in terms of intraluminal pressure changes and longitudinal muscle activity was analyzed a software-implemented process. Luminal volume output was measured using a photo-electric drop counter unit as described above.

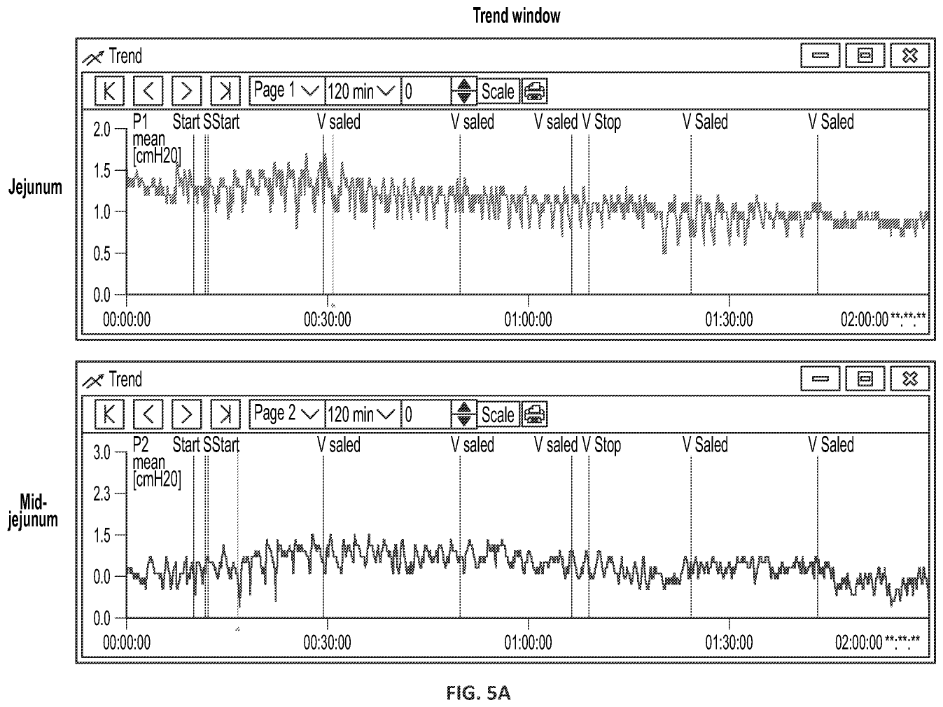

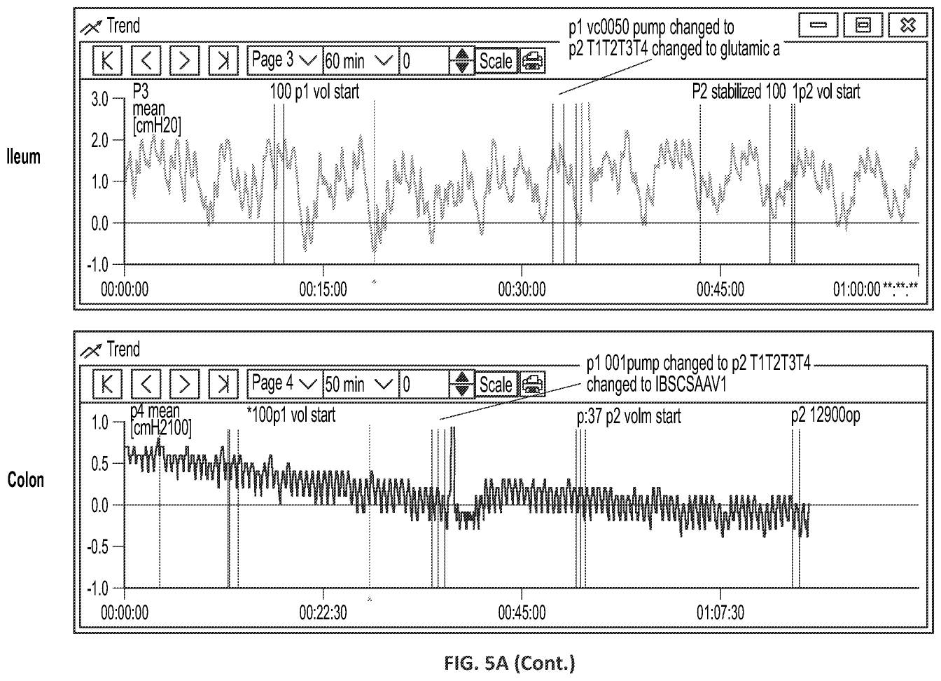

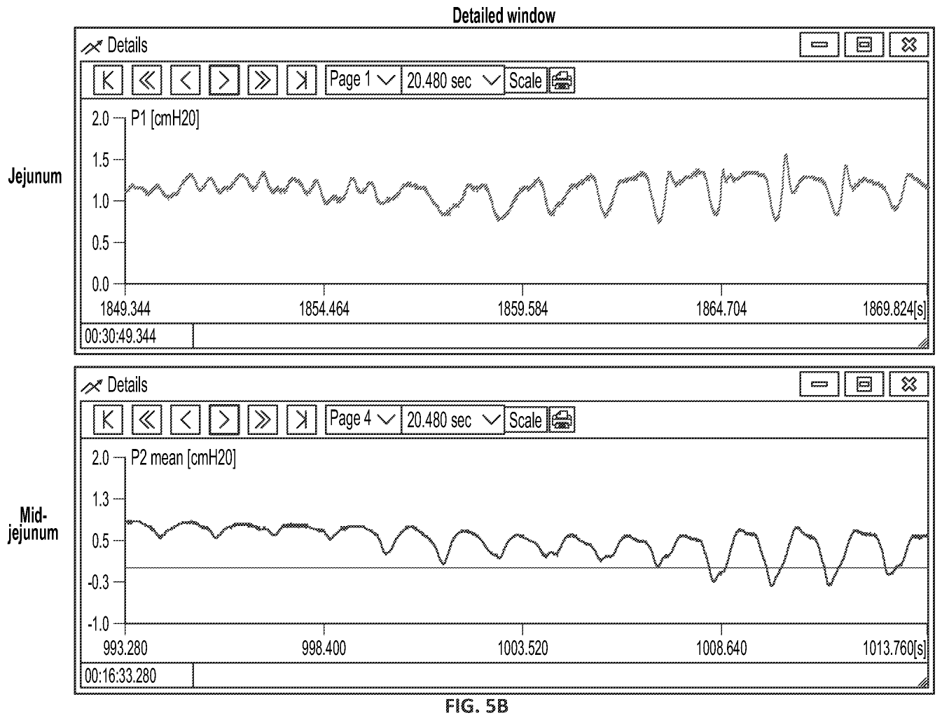

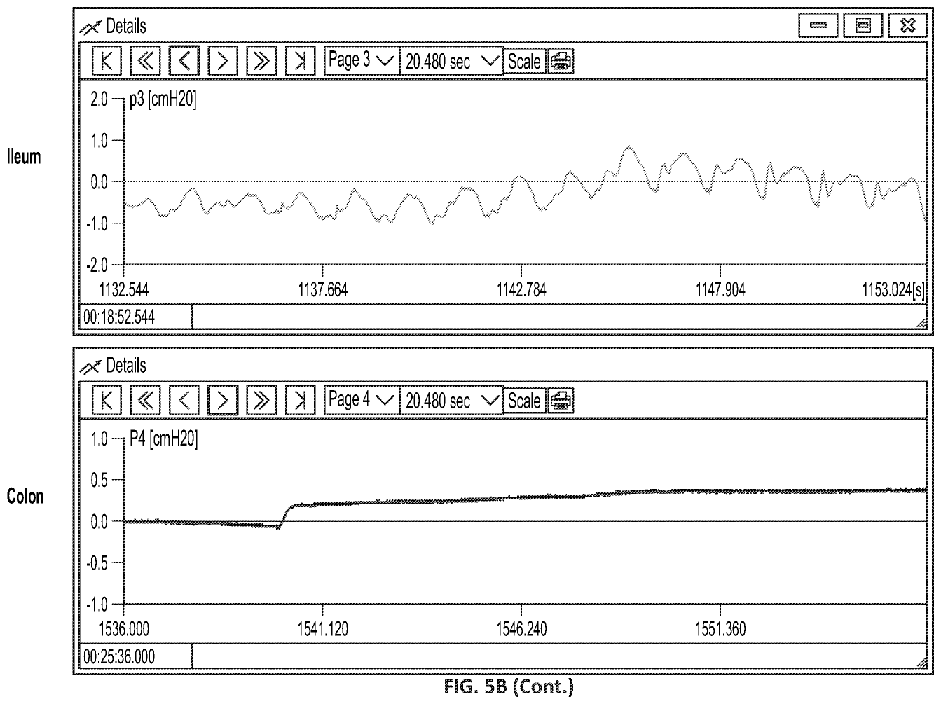

FIGS. 5A and 5B show graphs of experimental intraluminal pressure (ILP) pressure data recorded using a pressure transducer as described above and as captured using BDAS. As described above, pressure transducers were connected to the oral and aboral end of intestinal segments mounted in tissue baths. FIG. 5A shows trend view graphs of ILP data recorded in an experimental jejunum, mid-jejunum, ileum, and colon. FIG. 5B shows detailed view graphs of ILP changes in the same experimental jejunum, mid-jejunum, ileum, and colon.

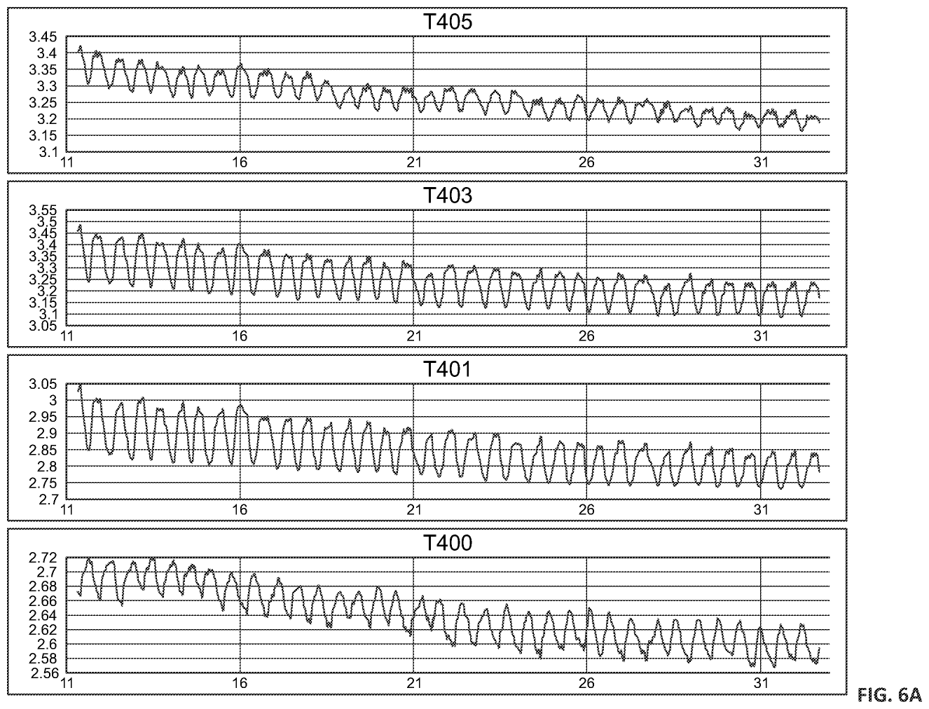

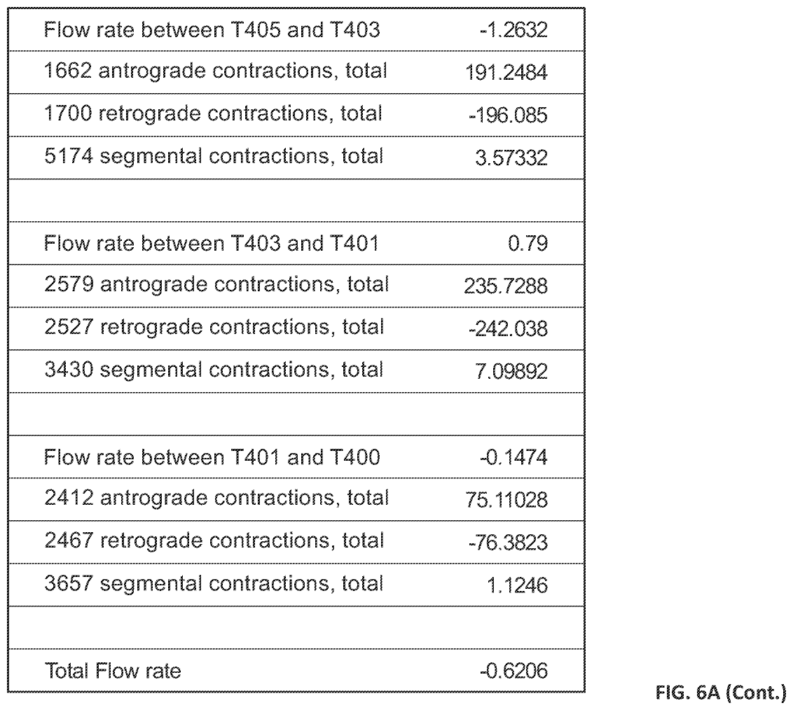

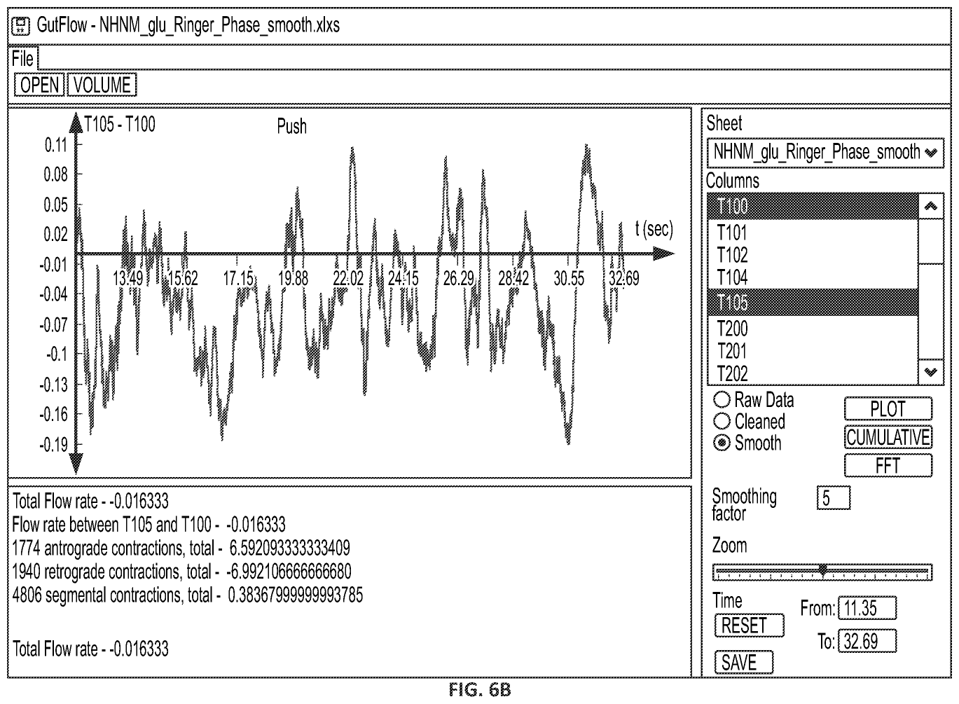

FIG. 6A shows representative graphs for diameter changes at different trackers in one of the tissue segments, which are utilized for calculating anterograde, segmental and retrograde contractions. FIG. 6B shows a screenshot of the data output from the program for anterograde-retrograde movement calculation.

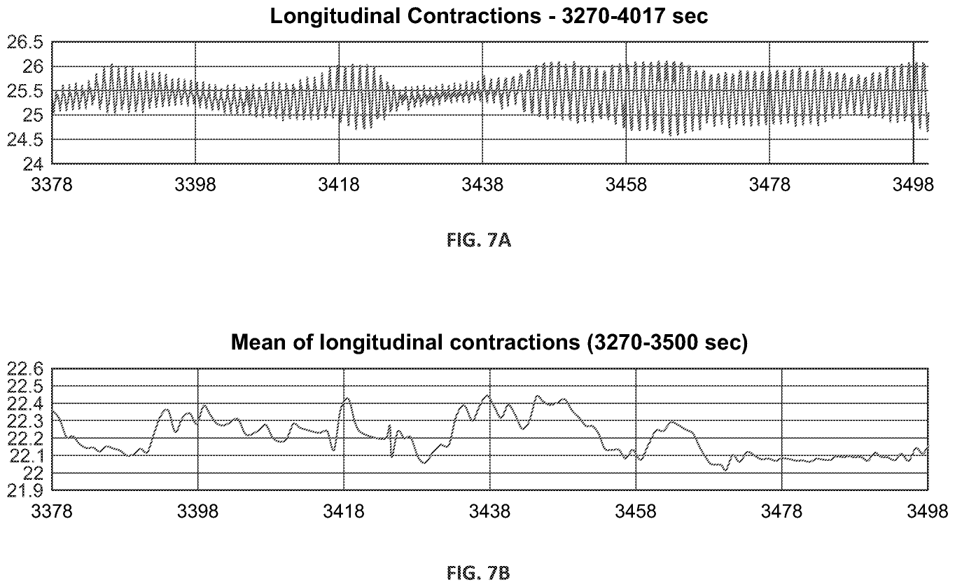

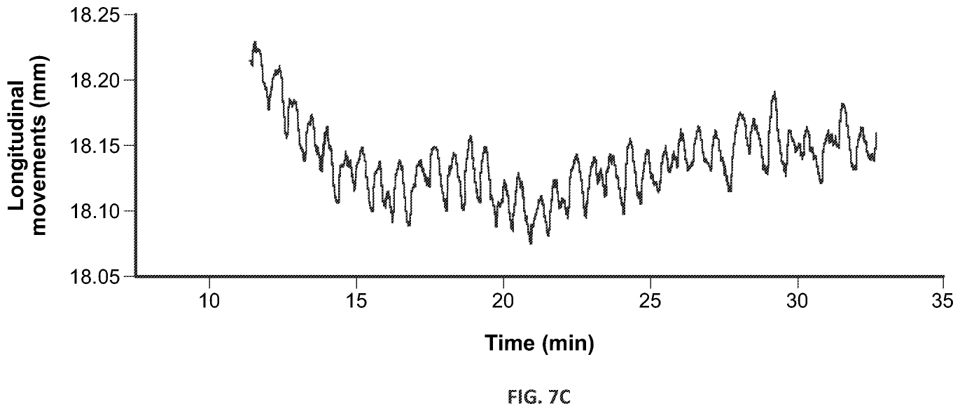

FIGS. 7A, 7B, and 7C show longitudinal movement of the intestinal segment, as measured with the tracker as described above, with respect to time. FIG. 7A shows a graph showing detailed changes in longitudinal contractions as used for frequency calculations. FIG. 7B shows a graph of mean changes in the longitudinal contractions representing the wave trends. FIG. 7C shows changes in longitudinal contraction over an extended period of time. In this graph, an upward shift suggests contraction.

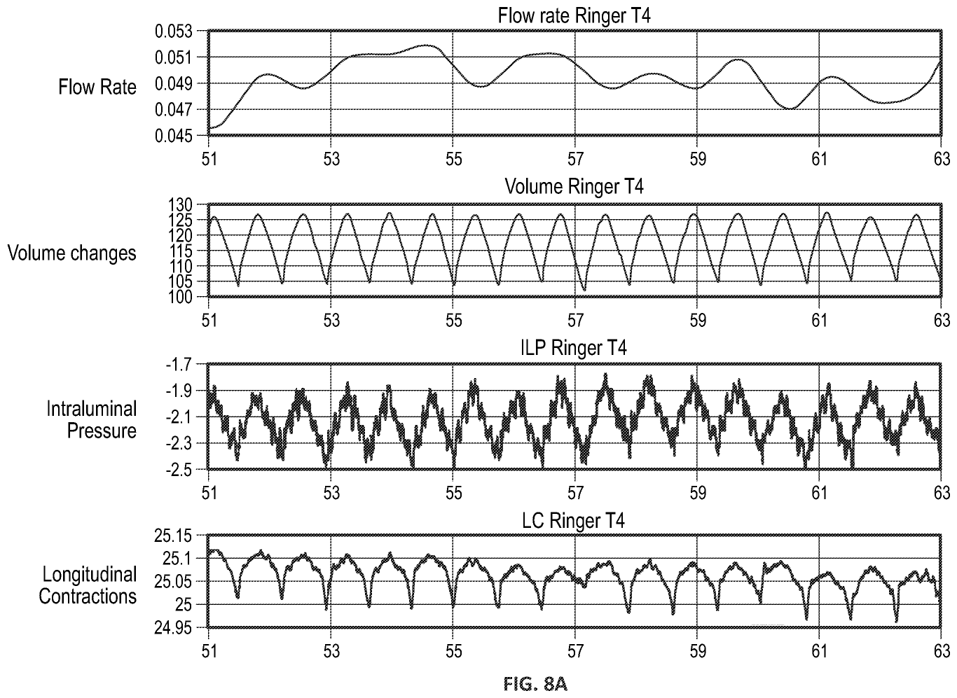

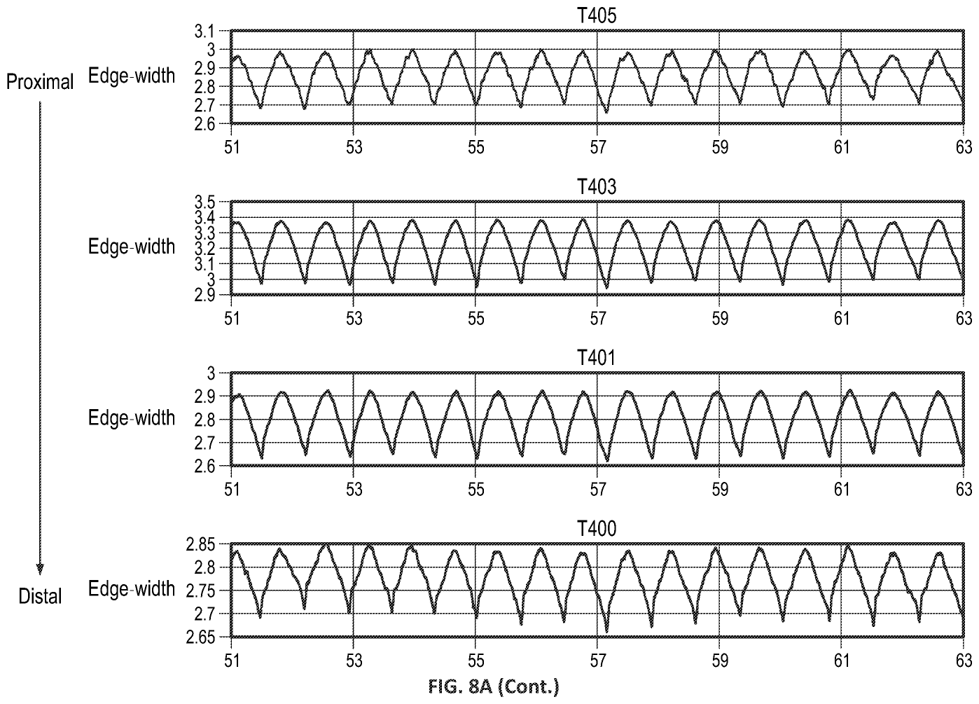

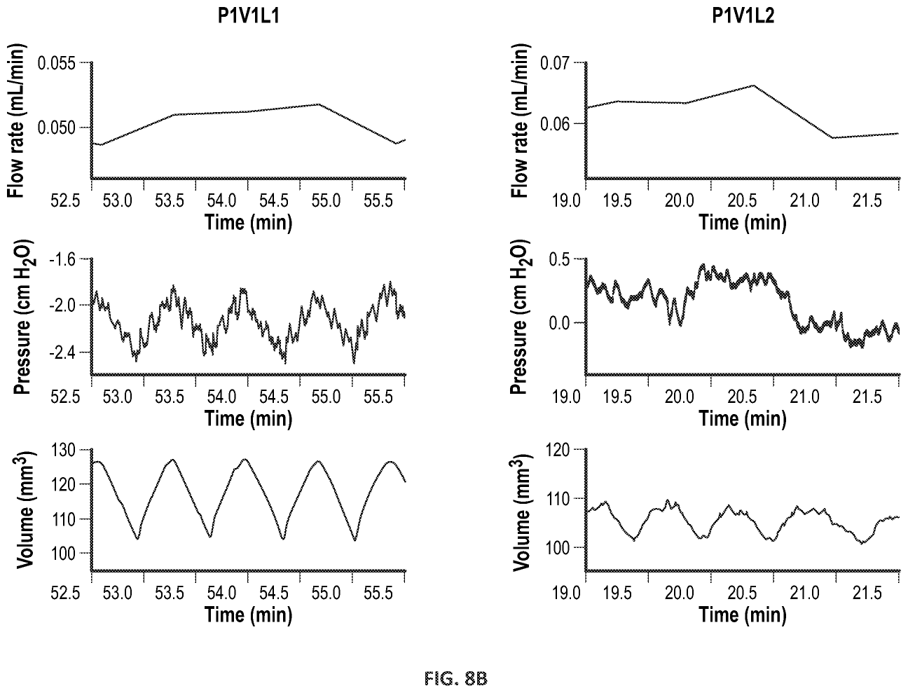

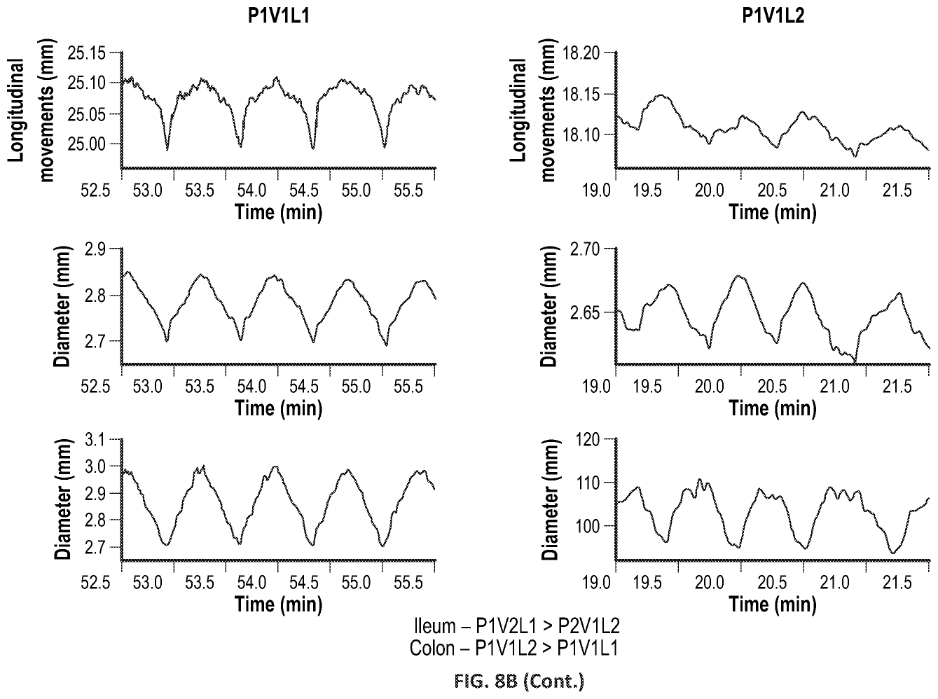

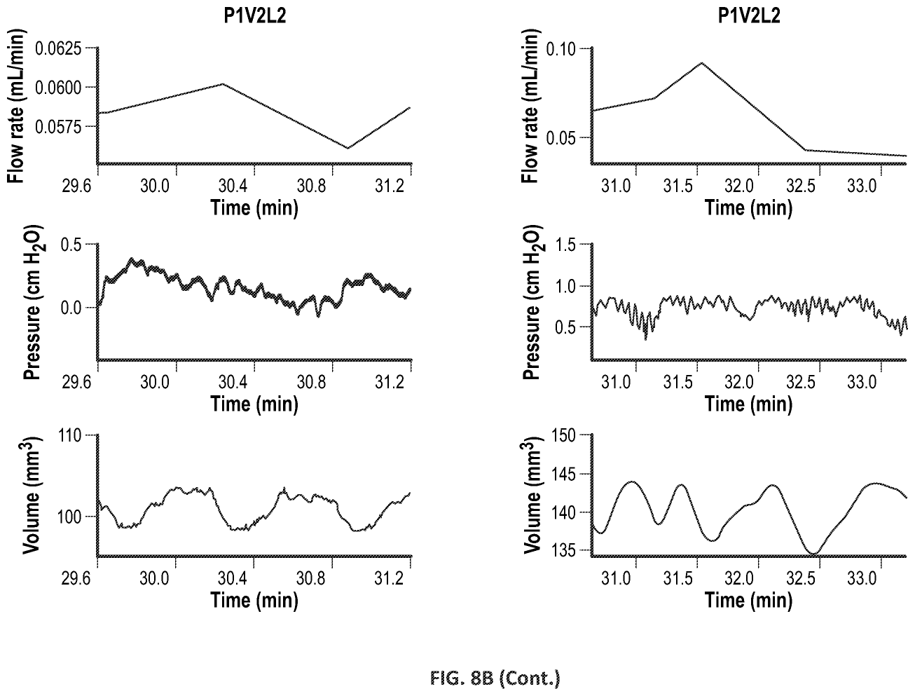

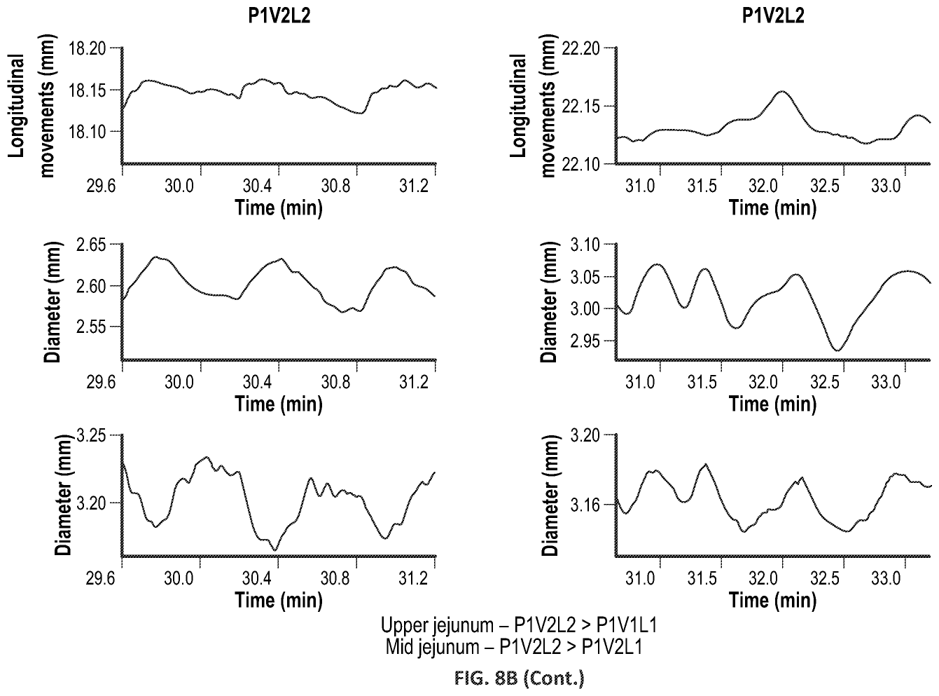

FIG. 8A shows graphs showing correlation between fluid output, intraluminal volume, ILP, and longitudinal contractions for the duration of the study. In particular, FIG. 8A shows a comparison of timings of droplet formation (in minutes) with changes in intraluminal volume (V), intraluminal pressure (P), longitudinal contractions (L) and diameter contractions (as shown by edge width data for proximal to distal trackers). As indicated in FIG. 8A, tracker T405 is the proximal most and T400 is the distal most tracker.

FIG. 8B shows graphs showing details in the changes in wave pattern in pressure, volume and longitudinal contractions to determine their relationship to outflow with narrow peaks and/or wide/broad peaks. In particular, FIG. 8B represents a comparison of all captured parameters for the time interval between each drop formation. As represented in FIG. 8B, an increase in P, V or L compared to a previous drop formation is represented as P1, V1 or L1 and a decrease in P, V or L compared to the previous event is represented as P2, V2 or L2. (e.g., the designation “P1V1L1” indicates an increase in pressure, volume and longitudinal contraction, the designation “P2V1L2” indicated an outflow event occurring when the pressure decreased, volume increased, and longitudinal movements relaxed, etc.). In some cases, the frequency of type of these events varied between various segments studied. In some cases, P1V2L1 was the type which occurred most frequently in producing an increased outflow when all three tissues (e.g., ileum, colon, jejunum) are considered together.

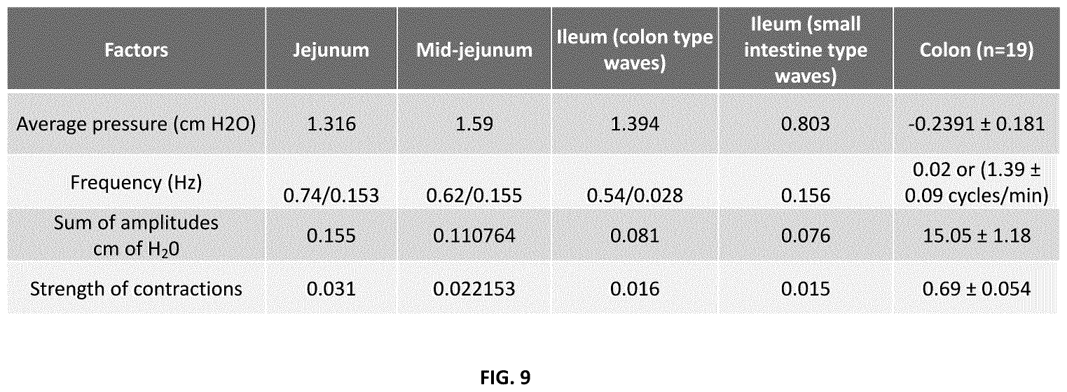

FIG. 9 shows a data table including average pressure in individual tissue segments as obtained from the BDAS software, as well as estimated values for frequency and amplitude of waves and strength of contractions based on the measured data. In particular, the specific values shown in FIG. 9 were obtained by use of the system 100 to evaluate physiological Ringer solution. In some embodiments, an output of data such as that shown in FIG. 9 can be obtained for a given formulation that is being tested using the system 100.

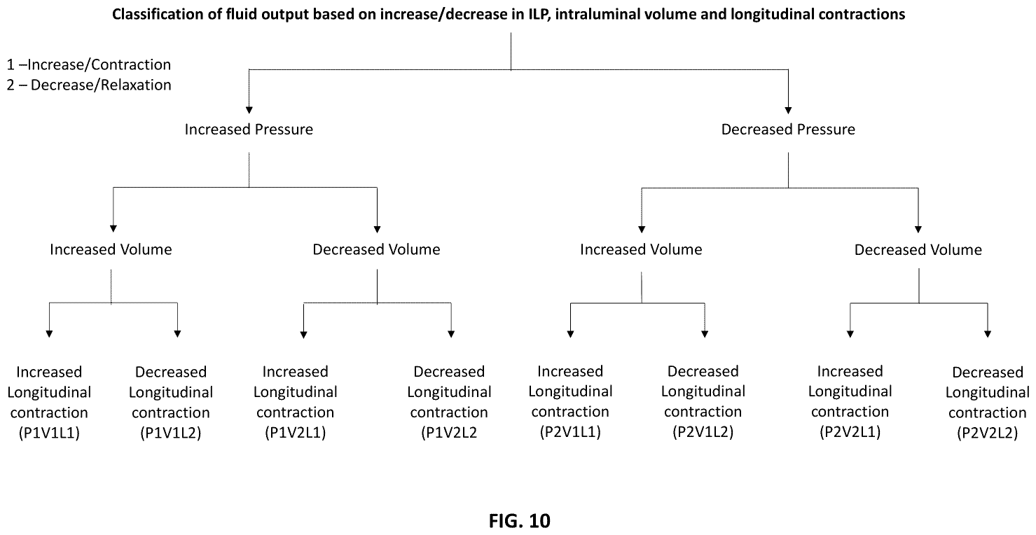

FIG. 10 shows categories for classification of a tested formulation based on increase/decrease in ILP, intraluminal volume and longitudinal contractions. In particular, FIG. 10 shows the categories into which tested formulation can be classified in accordance with the criteria described above with reference to FIG. 8B. In some embodiments, classification of a formulation in this manner may enable evaluation of the efficacy of tested formulations for treatment of certain symptoms, diseases, and/or conditions. For example, in some cases, where a certain category (e.g., P1V2L1) has been found to correlate with increased outflow, a formulation exhibiting a high frequency of this category may be evaluated as being suitable for treatment of constipation.

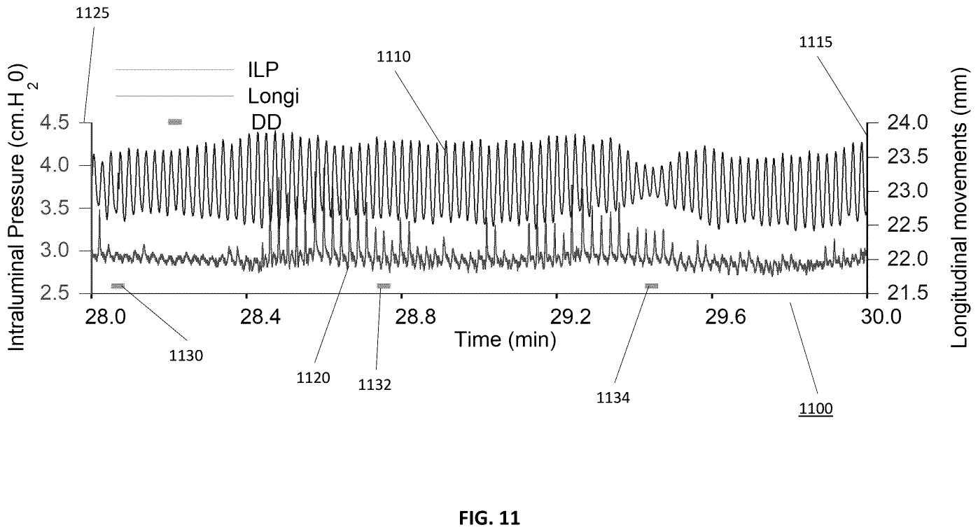

FIG. 11 shows a graph 1100 of data recorded using an experimental setup, including longitudinal muscle movements, intraluminal pressure, and droplet detection over a given time period. The graph 1100 shows a longitudinal muscle movement trace 1110 plotted against a longitudinal muscle movement axis 1115. The graph 1100 also shows an intraluminal pressure trace 1120 plotted against an intraluminal pressure axis 1125. The graph 1100 also shows times 1130, 1132, 1134 at which outflow droplets were detected. The graph 1100 shown in FIG. 11 demonstrates the correlation between longitudinal muscle movements, intraluminal pressure, and droplet formation.

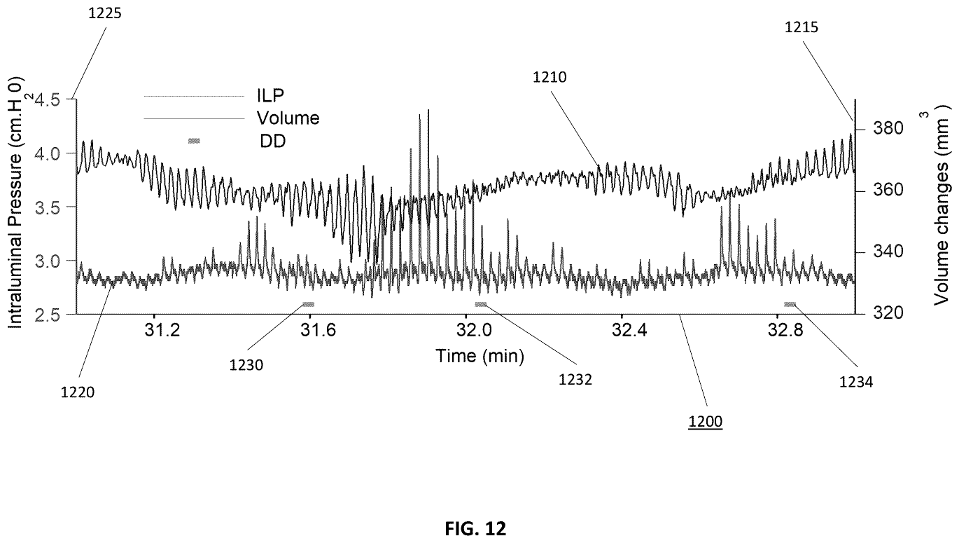

FIG. 12 shows a graph 1200 of data recorded using an experimental setup, including intraluminal volume, intraluminal pressure, and droplet detection over a given time period. The graph 1200 shows an intraluminal volume trace 1210 plotted against an intraluminal volume 1215. The graph 1200 also shows an intraluminal pressure trace 1220 plotted against an intraluminal pressure axis 1225. The graph 1200 also shows times 1130, 1132, 1134 at which outflow droplets were detected. The graph 1200 shown in FIG. 12 demonstrates the correlation between intraluminal volume, intraluminal pressure, and droplet formation.

Results:

The number, amplitude, duration, and frequency of high and low amplitude changes in ILP and longitudinal muscle activity were determined using custom written algorithm-based programs. The frequency of longitudinal muscle activity correlated well with the frequency of low and high amplitude pressure changes. The frequency of each of these oscillations was determined to be 0.7 Hz. A reduction in the ILP was found to induce high amplitude contractions which subsided as the ILP increased. Changes in intestinal diameter with time correlated to ILP changes. A reduction in luminal diameter caused an increase in ILP and when not fully compensated, caused increased fluid output. The mean velocity of the contractions over time in the regions tracked was determined to be 2.5-2.7 mm/sec with peak anterograde contraction velocity of −4.06 mm/sec and retrograde contractions of 12 mm/sec. This mean velocity in the presence of physiological Ringer's solution determines the direction of contractions along the length of the GI tract under normal conditions.

A common pacemaker activity was suggested by the discovery that a similar frequency was observed for longitudinal muscle activity and ILP oscillation. The ability to accurately measure the velocity of the contraction and volume output over time and correlate these values in an interpretable cohesive readout advances the field's understanding of overall peristaltic movement and thereby provides information that can be used as an indicator to gauge constipation and evaluate candidate agents for therapeutic efficacy in the context of conditions associated with constipation such as IBS-C.

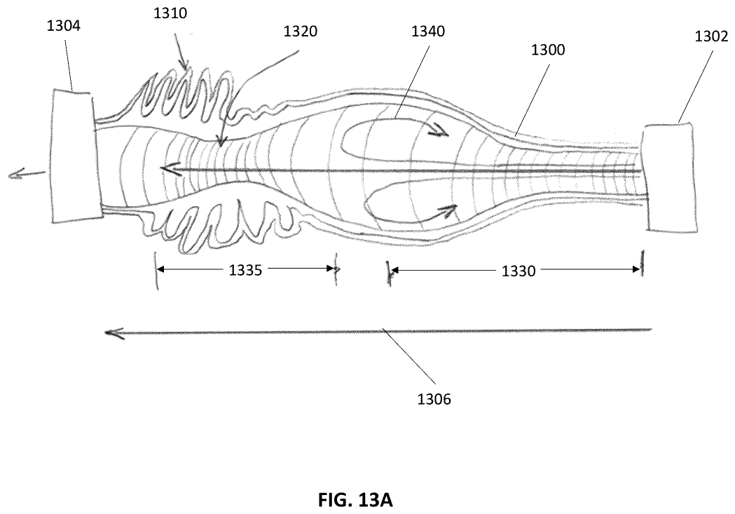

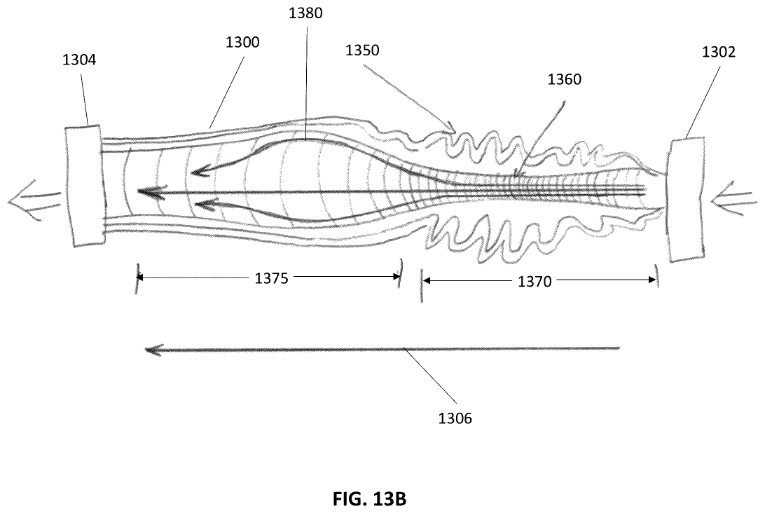

The data obtained through the use of the exemplary devices, systems, and methods may enable the behavior of the intestinal muscles (e.g., the longitudinal muscles and circular muscles) during peristalsis to be understood in a conceptual manner. FIGS. 13A and 13B show drawings of a representative segment of intestine 1300 in respective first and second stages of peristalsis. The intestine 1300 has a proximal end 1302 and a distal end 1304 defining a direction of propulsion 1306. FIG. 13A shows a first stage of peristalsis in the intestine 1300. As shown in FIG. 13A, a longitudinal muscle contraction 1310 and a circular muscle gathering 1320 are occurring near the distal end 1304 of the intestine 1300, thereby defining an area of relaxation 1330 near the proximal end 1302 and an area of contraction 1335 near the distal end 1304. Relaxation and contraction in this manner thereby induces flow 1340 into the intestine 1300 from the proximal end 1302. FIG. 13B shows a second stage of peristalsis in the intestine 1300. As shown in FIG. 13B, a longitudinal muscle contraction 1350 and a circular muscle gathering 1360 are occurring near the proximal end 1302 of the intestine 1300, thereby defining a propulsive segment 1370 near the proximal end 1302 and a receiving segment 1375 near the distal end 1304. As a result, fluid flow 1380 along the intestine 1300 toward the distal end 1304 is induced.

Other Results Included:
Longitudinal muscle activity and ILP oscillations showed similar frequency suggesting a common pacemaker activity in the intestine
Increase in intraluminal pressure (P1) is required for high amplitude anterograde/retrograde longitudinal muscle activity
The net luminal flow could be passive or active based on the contribution of ILP and longitudinal muscle activity
The net anterograde/retrograde movement, the velocity of the contractions and the fluid output measured elucidate the mechanism behind peristalsis, which could potentially lead to the development of therapeutic agents for constipation including IBS-C

While a number of embodiments of the present invention have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

What is claimed is:

1. A system, comprising:
a tissue bath;
a portion of an intestine positioned within the tissue bath,
wherein the portion of the intestine has a first end and a second end, and
wherein the portion of the intestine extends in a longitudinal direction from the first end to the second end;
a pump coupled to the first end of the portion of the intestine and configured to pump a fluid through the portion of the intestine;
a light source positioned to illuminate the portion of the intestine;
a camera positioned to capture a stream of images showing movement of the portion of the intestine when illuminated by the light source; and
a computing system coupled to the camera,
wherein the computing system includes:
a processor, and
a non-transitory memory storing instructions which, when executed by the processor, cause the processor to:
receive the stream of images,
identify a location along the portion of the intestine in each image of the stream of images in real time, and
measure an edge width at the location along the portion of the intestine in each image of the stream of images in real time.

2. The system of claim 1, wherein the tissue bath contains an isotonic solution.

3. The system of claim 1, the light source is configured to emit a green light.

4. The system of claim 1, wherein the camera has a resolution of at least 10 megapixels.

5. The system of claim 1, wherein the pump is a peristaltic pump.

6. The system of claim 5, wherein the peristaltic pump is configured to provide a flow rate that is in a range of from 0.001 milliliters per minute to 0.2 milliliters per minute.

7. The system of claim 1, further comprising a pressure transducer that is coupled to the portion of the intestine so as to measure an intraluminal pressure within the portion of the intestine.

8. The system of claim 1, further comprising a flow rate sensor that is coupled to the second end of the portion of the intestine so as to measure an outflow rate.

9. The system of claim 1, wherein the portion of the intestine is a portion of a mammal intestine.

10. The system of claim 1, wherein the instructions, when executed by the processor, cause the processor to execute pattern tracking software to measure the edge width.

11. The system of claim 1, wherein the instructions, when executed by the processor, further cause the processor to identify longitudinal contractions within the portion of the intestine.

12. A system, comprising:
a tissue bath;
a portion of an intestine positioned within the tissue bath,
wherein the portion of the intestine has a first end and a second end, and
wherein the portion of the intestine extends in a longitudinal direction from the first end to the second end;
a pump coupled to the first end of the portion of the intestine and configured to pump a fluid through the portion of the intestine;
a light source positioned to illuminate the portion of the intestine;
at least one camera positioned to capture a corresponding at least one stream of images showing movement of the portion of the intestine when illuminated by the light source;
a pressure transducer coupled to the portion of the intestine so as to detect an intraluminal pressure within the portion of the intestine; and
a computing system coupled to the at least one camera and to the pressure transducer,
wherein the computing system includes:
a processor, and
a non-transitory memory storing instructions which, when executed by the processor, cause the processor to:
receive the at least one stream of images,
identify a plurality of locations along the portion of the intestine in each image of the at least one stream of images in real time,
measure an edge width at each of the plurality of locations along the portion of the intestine in each image of the stream of images in real time; and
identify contractions in the portion of the intestine based on the measured edge widths at each of the plurality of locations.

13. The system of claim 12, wherein the light source is configured to emit a green light.

14. The system of claim 12, wherein the camera has a resolution of at least 10 megapixels.

15. The system of claim 12, further comprising a pressure transducer that is coupled to the portion of the intestine so as to measure an intraluminal pressure within the portion of the intestine.

16. The system of claim 12, further comprising a flow rate sensor that is coupled to the second end of the portion of the intestine so as to measure an outflow rate.

17. The system of claim 12, wherein the portion of the intestine is a portion of a mammal intestine.

18. The system of claim 12, wherein the contractions include at least one of longitudinal contractions or circular contractions.

19. A method, comprising:
operating a system including:
a tissue bath;
a portion of an intestine positioned within the tissue bath,
wherein the portion of the intestine has a first end and a second end, and
wherein the portion of the intestine extends in a longitudinal direction from the first end to the second end;
a pump coupled to the first end of the portion of the intestine and configured to pump a fluid through the portion of the intestine;
a light source positioned to illuminate the portion of the intestine;
at least one camera positioned to capture a corresponding at least one stream of images showing movement of the portion of the intestine;
a pressure transducer coupled to the portion of the intestine so as to measure an intraluminal pressure within the portion of the intestine; and
a computing system coupled to the at least one camera and to the pressure transducer, wherein the computing system includes:
a processor, and
a non-transitory memory storing instructions which, when executed by the processor, cause the processor to:
receive the at least one stream of images,
identify a plurality of locations along the portion of the intestine in each image of the at least one stream of images in real time,
measure an edge width at each of the plurality of locations along the portion of the intestine in each image of the stream of images in real time; and
identify contractions in the portion of the intestine based on the measured edge widths at each of the plurality of locations;
providing a first formulation,
wherein the first formulation comprises an active ingredient in a solution;
utilizing the pump to pump the first formulation through the portion of the intestine;
receiving, by the computing system, the at least one stream of images showing movement of the portion of the intestine while the first formulation is pumped through the portion of the intestine;
identifying, by the computing system, the plurality of locations along the portion of the intestine in each image of the at least one stream of images in real time;
measuring, by the computing system, the edge width at each of the plurality of locations along the portion of the intestine in each image of the stream of images in real time;
identifying, by the computing system, contractions in the portion of the intestine based on the measured edge widths at each of the plurality of locations;
determining at least one metric associated with the contractions;
obtaining at least one benchmark metric associated with a second formulation; and
determining an efficacy of the first formulation based on a comparison of the at least one metric to the at least one benchmark metric.

20. The method of claim 19, wherein the metric is a change in net anterograde/retrograde movement.

21. A method comprising:
receiving, by at least one processor, a plurality of images from at least one image sensor,
wherein the plurality of images depicts movement of an intestinal segment at a plurality of times;
receiving, by the at least one processor, a plurality of pressure measurements from at least one pressure transducer associated with the intestinal segment,
wherein the plurality of pressure measurements correspond to the plurality of times;
determining, by the at least one processor, a pattern of fluid flow through the intestinal segment based at least in part on the plurality of images and the plurality pressure measurements,
wherein the pattern of fluid flow comprises at least one of:
at least one first metric for a direction of the fluid flow, or
at least one second metric for a volume of the fluid flow;
determining, by the at least one processor, at least one treatment protocol based on the pattern of fluid flow; and
generating, by the at least one processor, at least one recommendation to a healthcare provider for the at least one treatment protocol.
